(19) United States
(12) Patent Application Publication
(10) Pub. No.: US 2023/0115572 A1
(43) Pub. Date: Apr. 13, 2023

(54) POST-OPERATIVE SURGICAL SITE WOUND TREATMENT AND METHOD FOR DEVICE REMOVAL

(71) Applicant: COR MEDICAL VENTURES, INC., Del Mar, CA (US)

(72) Inventors: Colin Murphy, San Diego, CA (US); Brian Bowman, Carlsbad, CA (US); Benjamin Arnold, San Diego, CA (US); Diana Rosales, Union City, CA (US); Conner Magnuson, San Diego, CA (US); Vikas Patel, Denver, CO (US); Christopher Kleck, Englewood, CO (US)

(21) Appl. No.: 17/802,842

(22) PCT Filed: Feb. 26, 2021
(86) PCT No.: PCT/US2021/019852

§ 371 Date: Aug. 26, 2022

Related U.S. Application Data
(60) Provisional application No. 62/982,653, filed on Feb. 27, 2020.

(51) Int. Cl. A61M 1/00

(52) U.S. Cl. CPC A61M 1/912

(57) ABSTRACT

A device and method for treatment of post-operative surgical site wounds. The device includes a housing, a spool configured to rotate relative to the housing, and a catheter coupled to the spool. Rotating the spool in one direction winds the catheter about a column disposed on the spool, and enables the catheter to be retracted from the closed wound. In some embodiments, the device further includes a wound dressing disposed about a distal end of the catheter. In some embodiments, retracting the catheter from a closed wound enables a wound dressing to be retracted from the closed wound.

### CROSS-REFERENCES TO RELATED APPLICATIONS

This application claims the benefit of U.S. Provisional Patent Application No. 62/982,653 filed Feb. 27, 2020, the disclosure of which is incorporated herein by reference.

### BACKGROUND OF THE INVENTION

Surgical drains are often used to treat deep post-operative wounds. Surgical drains are tubes commonly placed by surgeons to remove blood, pus, and infected fluids from a wound bed and to prevent the accumulation of air, or formation of dead space. If allowed to accumulate, the fluids or air can put pressure on the surgical site and adjacent areas, causing pain. There are several types of surgical drains including open or closed, and suction or passive drains. Closed-suction drains are maintained under low or high pressure to create a suction and drain into a bag or bottle.

The general surgical drain protocol involves inserting a drain into the wound and attaching the external end to a suction source. The negative pressure from the suction source removes exudates from the cavity. The wound can be closed around the drain. Surgical drains may fail to suction over the entire wound area and when removed, often still leave a relatively large dead space.

Negative-pressure wound therapy (NPWT) is used to promote healing in acute or chronic wounds. A sealed wound dressing fills the wound, attaching to a vacuum pump to apply reduced pressure, such as sub-atmospheric pressure. The vacuum acts as a suction device, removing fluid from the wound and drawing the edges of the wound inward. Additionally, sub-atmospheric pressure assists in wound closure by promoting blood flow to the area and stimulating granulation tissue formation. NPWT systems can also provide irrigation or antibiotics to the wound.

The general NPWT protocol involves placing the wound dressing in the wound and placing a flexible cover layer over the wound to create a seal, or vacuum reservoir, where the reduced pressure can be applied. The NPWT tubing can then be attached to the wound dressing at the skin level with an adhesive transfer pad.

NPWT systems can also be used for deep post-operative wound closure. The wound dressing is packed into the deep wound and is attached to a suction drainage system that can provide irrigation and remove exudates through the negative pressure system. The wound can be closed temporarily but a removal surgery is necessary to remove the dressing from the wound, leaving a relatively large dead space.

Therefore, there is a need for improved surgical site wound management devices, systems and methods that overcome some of the challenges associated with commercially available technology. The present disclosure describes improvements over existing technologies to allow for more complete post-operative surgical site wound management.

### SUMMARY OF THE INVENTION

The present disclosure generally relates to medical devices, systems and methods and more particularly relates to post-operative wound closure devices, systems and treatment methods.

A combination of closed-suction drains and a NPWT system for deep post-operative wound closure can be beneficial. It may be desirable to have a system that includes drains to reach deep into surgical wounds with NPWT dressings at the wound surface. It may be beneficial if the drain could provide suction. It may be beneficial for the NPWT dressing to help close the wound. It may be beneficial for the wound to be closed around the drain. It may be desirable for devices, systems, and methods to eliminate the problem of the drain leaving a relatively large space between tissues that are normally anatomically connected (e.g. dead space). It may be desirable for devices, systems, and methods to eliminate the problem of the drain failing to provide a large surface area over which suction and/or irrigation can occur. Such a device may be beneficial for infection cases, spine surgeries, orthopedic fracture surgeries, plastic surgeries, or any other surgeries.

It may be desirable to provide deep-wound suction over a greater area. It may be desirable to provide complete wound irrigation at a local level. It may be desirable to deliver high-concentrations of local antibiotics to deep wounds. It may be desirable to promote wound closure with negative pressure. It may be desirable to treat post-operative wounds through a NPWT system without having to re-open the wound for component removal.

Any embodiment of the device may comprise an elongate shaft. Any embodiment of the device may comprise a wound dressing. Any embodiment of the device may comprise a removal element. The elongate shaft is preferably hollow, such as a tube, cannula, or catheter. The elongate shaft may have an external end disposed outside the patient and an internal end disposed in the patient's wound. The wound dressing may be an open pore, reticulated, hydrophobic foam sponge or may take any other form known in the art. The removal element may be a braid, cage, radially expandable member, or take any other form. The removal element may have any number of patterns but in preferred embodiments may be a helical braid with a distal end that is disposed in the wound and a trailing end that extends proximally outward toward a location external of the patient, away from the wound. The removal element may be collapsible. The distal end of the removal element may be of diameter or width greater than the elongate hollow tube in its expanded state. The distal end of the removal element may be of a diameter or width less than the lumen of the elongate hollow tube in its collapsed state. The distal end of the removal element may be a bulbous shape, cylindrical shape, or any other shape. The distal end of the removal element may conform to the contour of the wound dressing. The wound dressing may expand until constrained by the shape of the removal element. The internal end of the elongate shaft may abut the wound dressing. The removal element components may join to make up the trailing end. The trailing end of the removal element may span the length of the lumen of the elongate shaft, through the external end disposed outside the patient. The external end of the elongate shaft may connect to a suction and/or irrigation system with a fitting. The suction and/or irrigation system may be a NPWT system. The fitting may be a Luer lock or a custom fitting.

Optionally in any embodiment, the elongate shaft may have a y-split near the external end disposed outside the patient's wound, with two external arms. The first external arm end may connect to a suction and/or irrigation system with a fitting. The suction and/or irrigation system may be a NPWT system. The fitting may be a Luer lock or a custom fitting. A stopper of cylindrical shape may have a tight fit with the lumen of the elongate shaft. The stopper may have O-rings sized to maintain the negative-pressure seal in the elongate shaft. The stopper may reside in the second external arm of the elongate shaft. The removal element trailing end may extend past the second external arm end disposed outside the patient. The stopper may capture the trailing end of the removal element. The removal element may be captured by molding processes, mechanical mechanisms, bonding, or any other processes. The removal element may be pulled axially, causing the stopper to move accordingly.

Optionally in any embodiment, the elongate shaft may have a y-split near the external end disposed outside the patient's wound, with two external arms. One external arm end may connect to a suction and/or irrigation system with a fitting. The suction and/or irrigation system may be a NPWT system. The fitting may be a Luer lock or a custom fitting. An externally threaded cylindrical fitting may be bonded to the second external arm end. A cylindrical cap may have internal threads that mate with those of the fitting. The cap and the fitting threads may mate to maintain the negative-pressure seal in the elongate shaft. The removal element trailing end may extend into the second external arm of the elongate shaft, connecting to the threaded cap. The trailing end may connect to the threaded cap by molding processes, mechanical mechanisms, bonding, or any other processes. The cap may be unthreaded from the fitting, such that the removal element is pulled axially.

Optionally in any embodiment, the elongate shaft may have a y-split near the external end disposed outside the patient's wound, with two external arms. One external arm end may connect to a suction and/or irrigation system with a fitting. The suction and/or irrigation system may be a NPWT system. The fitting may be a Luer lock or a custom fitting. The outer diameter of the second external arm end may have cylindrical ratchets. The ratchets may be molded onto the elongate shaft or may be a fitting bonded onto the elongate shaft. A cylindrical cap may have a sliding fit with the ratcheted fitting. The cap may have an actuation mechanism that interfaces with the fitting ratchets. The actuation mechanism may be pushed to release the ratcheting to allow for axial movement. The cap and the fitting may maintain the negative-pressure seal in the elongate shaft. The cap may be moved axially along the fitting, such that the removal element is pulled axially.

Optionally in any embodiment, the elongate shaft may be multi-lumen. The elongate shaft may have one lumen that extends distally into the wound dressing. The extended lumen may have a closed tip or an open tip. The extended lumen may have a plurality of holes. The holes may be patterned axially and/or radially along the lumen. The holes may allow for removal of suctioned exudate along the length of the dressing. The holes may allow for distribution of irrigating fluids along the length of the dressing.

Optionally in any embodiment, the elongate shaft may be multi-lumen to provide separate pathways for suction and irrigation.

Optionally in any embodiment, there may be multiple elongate shafts that are connected along their length to provide separate pathways for irrigation and suction.

In some embodiments, there may be a series of wound dressings. The wound dressings may be captured in the removal element in order to fill a larger wound space.

Preferably the device may insert the wound dressing into the wound space of a clean wound for treatment. The elongate shaft may span from the wound bed to a suction and/or irrigation system disposed external of the patient. The wound may be closed around the elongate shaft. The device may be used with the external system to provide irrigation. The device may be used with the external system to suction exudates. The device may be used with the external system to instill antibiotics.

Preferably this device may remove the wound dressing after treatment through a closed-wound, without the need for a removal surgery. A removal mechanism may be used for device collapse and retraction. The trailing end of the removal element may be pulled axially, causing the distal end of the removal element to lengthen and narrow. The narrowed removal element may collapse the wound dressing. The trailing end of the removal element may continue to be pulled axially to retract the wound dressing into the elongate shaft. The device may be removed through a closed wound.

Optionally in any embodiment, the method for removal may include incremental collapse of the wound dressing with continued therapy.

Also provided herein is a post-operative surgical site wound treatment device, the device comprising: a housing comprising: an aperture in an outer surface of the housing; a channel extending from the aperture to an interior surface of the housing, wherein at least a portion of the channel is tapered; a spool configured to rotate relative to the housing, wherein at least a portion of the spool resides within the housing, the spool comprising: a column; a source opening on an outer surface of the spool; a catheter opening on an inner surface of the spool; and a conduit between the source opening and the catheter opening; and a catheter having a distal portion and a proximal portion, wherein the proximal portion wraps around the column of the spool, wherein a terminus of the proximal portion couples to the catheter opening of the spool, and wherein the distal portion is inserted through the aperture of the housing and into the closed wound.

In some embodiments, the taper is a linear taper. In some embodiments, the taper is a logarithmic taper In some embodiments, the taper tapers inwards from the aperture. In some embodiments, the taper has a minimum diameter of greater than an outer diameter of the catheter. In some embodiments, the housing further comprises a pressure outlet extending from the outer surface of the housing to the interior of the housing. In some embodiments, the pressure outlet is disposed proximal to the channel. In some embodiments, the pressure outlet is disposed on the channel. In some embodiments, the outer surface of the housing further comprises a flange. In some embodiments, flange is disposed about the aperture. In some embodiments, the outer surface of the spool comprises a torque grip. In some embodiments, the torque grip comprises a depression, a protuberance, or both. In some embodiments, the conduit is within the column. In some embodiments, the column and the conduit are concentric. In some embodiments, the column and the source opening are concentric. In some embodiments, the source opening, the catheter opening, or both comprise a luer connector. In some embodiments, at least one of the source opening and the catheter opening comprise a male luer connector, a female luer connector, or both. In some embodiments, at least one of the source opening and the catheter opening comprise two or more luer connectors. In some embodiments, the catheter comprises one or more perforations between an outer surface of the catheter and an inner surface of the catheter. In some embodiments, the one or more perforations are disposed about the distal portion of the catheter. In some embodiments, a flange in the spool, the housing, or both enables the spool to rotate relative to the housing. In some embodiments, a groove in the spool, the housing, or both enables the spool to rotate relative to the housing. In some embodiments, rotating the spool in a first direction relative to the housing winds the catheter onto the column, and wherein rotating the spool in a second direction opposite the first direction unwinds the catheter from the column. In some embodiments, rotating the spool in a first direction relative to the housing translates at least a portion of the catheter into the housing, and wherein rotating the spool in a second direction opposite translates at least a portion of the catheter out of the housing. In some embodiments, at least a portion of the spool contacts an inner surface of the housing when the spool rotates within the housing. In some embodiments, the device further comprises a wound dressing disposed about the distal portion of the catheter. In some embodiments, the device further comprises a mesh disposed about the wound dressing. In some embodiments, the catheter and the source opening are in two-way fluidic communication.

Another aspect provided herein is a method of post-operative surgical site treatment through a closed wound, the method comprising: providing the device of any claim; inserting the catheter into the wound; closing the wound about the catheter; and delivering an antibiotic and/or irrigation fluids, and/or suctioning exudates and/or other material from the closed wound.

In some embodiments, the antibiotic and/or irrigation fluids are delivered through the source opening. In some embodiments, exudates are suctioned through the source opening and/or the pressure outlet. In some embodiments, the method further comprises removing the catheter from the closed wound by rotating the spool. In some embodiments, the device comprises a wound dressing disposed about a first portion of the catheter, wherein the wound dressing and the first portion of the catheter are inserted into the wound. In some embodiments, the method further comprises removing the catheter and wound dressing from the closed wound by rotating the spool. In some embodiments, the method further comprises removing a material from the closed wound via a negative pressure source coupled to the source opening. In some embodiments, the method further comprises removing a material from the closed wound via a negative pressure source coupled to the pressure outlet. In some embodiments, the method further comprises removing exudates from the closed wound via the catheter. In some embodiments, the method further comprises delivering a material to the closed wound via the source opening. In some embodiments, material comprise antibiotics, irrigations fluid, or any combination thereof, to the closed wound.

Additional aspects of the invention will be apparent from the detailed descriptions and claims herein.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a side view of an exemplary embodiment of a device for post-operative wound treatment having a removal element that encompasses a sponge, in accordance with some embodiments;

FIG. 1B shows a side view of an exemplary embodiment of a device for post-operative wound treatment having a removal element that is woven through a sponge, in accordance with some embodiments;

FIG. 1C shows a side view of an exemplary embodiment of a device for post-operative wound treatment having radiopaque markers on components of the device, in accordance with some embodiments;

FIG. 2A shows a side view of an exemplary embodiment of the internal end of the elongate shaft, the internal end having a flare in accordance with some embodiments;

FIG. 2B shows a side view of an exemplary embodiment of the internal end of the elongate shaft, the internal end having an external chamfer in accordance with some embodiments;

FIG. 2C shows a side view of an exemplary embodiment of the internal end of the elongate shaft, the internal end having no flare in accordance with some embodiments;

FIG. 3A shows a side view of an exemplary embodiment of the removal element, in accordance with some embodiments;

FIG. 3B shows a close-up of an exemplary embodiment of the removal element, in accordance with some embodiments;

FIG. 4A shows a side view of another exemplary embodiment of a device for post-operative wound treatment with a y-split and stopper, in accordance with some embodiments;

FIG. 4B shows a cross-section of another exemplary embodiment of a device for post-operative wound treatment with a y-split and stopper, in accordance with some embodiments;

FIG. 4C shows a side view of another exemplary embodiment of a device for post-operative wound treatment with a y-fitting, in accordance with some embodiments;

FIG. 5 shows a cross-section view of an exemplary embodiment of a stopper, in accordance with some embodiments;

FIG. 6A shows a side view of another exemplary embodiment of a device for post-operative wound treatment with a y-split and threaded fitting, in accordance with some embodiments;

FIG. 6B shows a cross-section view of another exemplary embodiment of a device for post-operative wound treatment with a y-slit and threaded fitting, in accordance with some embodiments;

FIGS. 7A and 7B show a cross-section view of an exemplary embodiment of the external end threaded fitting and cap, in accordance with some embodiments;

FIG. 8 shows a side view of another exemplary embodiment of a device for post-operative wound treatment with y-split and ratcheted fitting, in accordance with some embodiments;

FIG. 9 shows a cross-section view of an exemplary embodiment of the external end ratcheted fitting and cap, in accordance with some embodiments;

FIG. 10 shows a cross-section of another exemplary embodiment with a multi-lumen elongate shaft, one lumen extending proximally into the wound dressing, in accordance with some embodiments;

FIG. 11 shows a side view of another exemplary embodiment of the elongate shaft with separate irrigation and suction channels, in accordance with some embodiments;

FIG. 12 shows a side view of another exemplary embodiment of the elongate shaft with separate irrigation and suction channels, in accordance with some embodiments;

FIG. 13 shows a side view of another exemplary embodiment of a series of wound dressings captured by the removal element, in accordance with some embodiments;

FIG. 14 shows a side view of another exemplary embodiment of a removal element woven into a wound dressing, in accordance with some embodiments;

FIG. 15 shows a side view of another exemplary embodiment of the removal element composed of hollow components, in accordance with some embodiments;

FIG. 16 shows a schematic of an exemplary embodiment of a device for post-operative wound treatment being connected to a suction and/or irrigation device, in accordance with some embodiments;

FIG. 17 shows a cross-section view of a device for post-operative wound treatment placed in a spinal wound, in accordance with some embodiments;

FIG. 18 shows a superior view of a device for post-operative wound treatment placed in a spinal wound, in accordance with some embodiments;

FIG. 19 shows a side view of the removal element being pulled axially, in accordance with some embodiments;

FIG. 20A shows a side view of the wound dressing beginning to collapse due to the lengthening and narrowing of the removal element, in accordance with some embodiments;

FIG. 20B shows a side view of the wound dressing having a sponge partially retracted in an initial position, in accordance with some embodiments;

FIG. 21A shows a cross-section view of a fully collapsed and retracted wound dressing into the elongate shaft, in accordance with some embodiments;

FIG. 21B shows a cross-section view of a fully collapsed and retracted wound dressing into the elongate shaft having an internal chamfer, in accordance with some embodiments;

FIG. 22 shows a side view of a device for post-operative wound treatment being removed out of a closed wound, in accordance with some embodiments;

FIG. 23 shows a front-right-top perspective view of a post-operative surgical site wound treatment device, in accordance with some embodiments;

FIG. 24 shows a bottom view of a post-operative surgical site wound treatment device, in accordance with some embodiments;

FIG. 25 shows a front-left-bottom perspective view of a post-operative surgical site wound treatment device, in accordance with some embodiments;

FIG. 26 shows a front-right-top perspective illustration of a post-operative surgical site wound treatment device, in accordance with some embodiments;

FIG. 27A shows a front illustration and FIG. 27B shows a top cross-sectioned illustration of a housing and spool for a post-operative surgical site wound treatment device, in accordance with some embodiments;

FIG. 28A shows a right cross-sectioned illustration and FIG. 28B shows a front illustration and a of a spool of the post-operative surgical site wound treatment device, in accordance with some embodiments;

FIG. 29A shows a top cross-sectioned illustration and FIG. 29B shows a front illustration of a housing of the post-operative surgical site wound treatment device, in accordance with some embodiments;

FIG. 30 shows a front cross-sectioned illustration of a catheter and wound dressing of the post-operative surgical site wound treatment device, in accordance with some embodiments;

FIG. 31 shows a front-top-right perspective illustration of a catheter of the post-operative surgical site wound treatment device, in accordance with some embodiments;

FIGS. 32A, 32B, and 32C show front, left, and back illustrations respectively of a catheter of the post-operative surgical site wound treatment device, in accordance with some embodiments;

FIG. 33 shows a detailed front-top-left perspective illustration of a catheter coupled to the catheter opening of the post-operative surgical site wound treatment device, in accordance with some embodiments;

FIG. 34 shows a first image of the post-operative surgical site wound treatment device, in accordance with some embodiments;

FIG. 35 shows a second image of the post-operative surgical site wound treatment device, in accordance with some embodiments;

FIG. 36 depicts aspects of a post-operative surgical site wound treatment device, in accordance with some embodiments;

FIG. 37 depicts aspects of a post-operative surgical site wound treatment device, in accordance with some embodiments;

FIG. 38 depicts aspects of a post-operative surgical site wound treatment device, in accordance with some embodiments;

FIG. 39 depicts aspects of a post-operative surgical site wound treatment device, in accordance with some embodiments;

FIG. 40 depicts aspects of a post-operative surgical site wound treatment device, in accordance with some embodiments;

FIG. 41 depicts aspects of an incision site mechanism of a post-operative surgical site wound treatment device, in accordance with some embodiments;

FIG. 42 depicts aspects of a spool of a post-operative surgical site wound treatment device, in accordance with some embodiments;

FIG. 43 depicts aspects of a spool of a post-operative surgical site wound treatment device, in accordance with some embodiments;

FIG. 44 depicts aspects of an incision site mechanism of a post-operative surgical site wound treatment device, in accordance with some embodiments;

FIG. 45 depicts aspects of a spool lid of a post-operative surgical site wound treatment device, in accordance with some embodiments;

FIG. 46 depicts aspects of a spool lid of a post-operative surgical site wound treatment device, in accordance with some embodiments;

FIG. 47 depicts aspects of a spool column of a post-operative surgical site wound treatment device, in accordance with some embodiments;

FIG. 48 depicts aspects of a spool column of a post-operative surgical site wound treatment device, in accordance with some embodiments;

FIG. 49 depicts aspects of a spool lever of a post-operative surgical site wound treatment device, in accordance with some embodiments;

FIG. 50 depicts aspects of a spool lever of a post-operative surgical site wound treatment device, in accordance with some embodiments;

FIG. 51 depicts aspects of a spool housing of a post-operative surgical site wound treatment device, in accordance with some embodiments;

FIG. 52 depicts aspects of a spool housing of a post-operative surgical site wound treatment device, in accordance with some embodiments;

FIG. 53 depicts aspects of an incision site connector of a post-operative surgical site wound treatment device, in accordance with some embodiments;

FIG. 54 depicts aspects of an incision site connector of a post-operative surgical site wound treatment device, in accordance with some embodiments;

FIG. 55 depicts aspects of a pin of a post-operative surgical site wound treatment device, in accordance with some embodiments;

FIG. 56 depicts aspects of a luer fitting (e.g. female) of a post-operative surgical site wound treatment device, in accordance with some embodiments;

FIG. 57 depicts aspects of a vacuum hose fitting of a post-operative surgical site wound treatment device, in accordance with some embodiments;

FIG. 58 depicts aspects of a vacuum hose fitting of a post-operative surgical site wound treatment device, in accordance with some embodiments;

FIG. 59 depicts aspects of a C-clip of a post-operative surgical site wound treatment device, in accordance with some embodiments;

FIG. 60 depicts aspects of an O-ring of a post-operative surgical site wound treatment device, in accordance with some embodiments;

FIG. 61 depicts aspects of a foam assembly of a post-operative surgical site wound treatment device, in accordance with some embodiments;

FIG. 62 depicts aspects of a foam assembly of a post-operative surgical site wound treatment device, in accordance with some embodiments;

FIG. 63 depicts aspects of a foam assembly of a post-operative surgical site wound treatment device, in accordance with some embodiments;

FIG. 64 depicts aspects of a male luer connector of a post-operative surgical site wound treatment device, in accordance with some embodiments;

FIG. 65 depicts aspects of a male luer connector of a post-operative surgical site wound treatment device, in accordance with some embodiments;

FIG. 66 depicts aspects of a female luer connector of a post-operative surgical site wound treatment device, in accordance with some embodiments; and

FIG. 67 depicts aspects of a female luer connector of a post-operative surgical site wound treatment device, in accordance with some embodiments.

### DETAILED DESCRIPTION OF THE INVENTION

Specific embodiments of the disclosed device, system and method of use will now be described with reference to the drawings. Nothing in this detailed description is intended to imply that any particular component, feature, or step is essential to the invention.

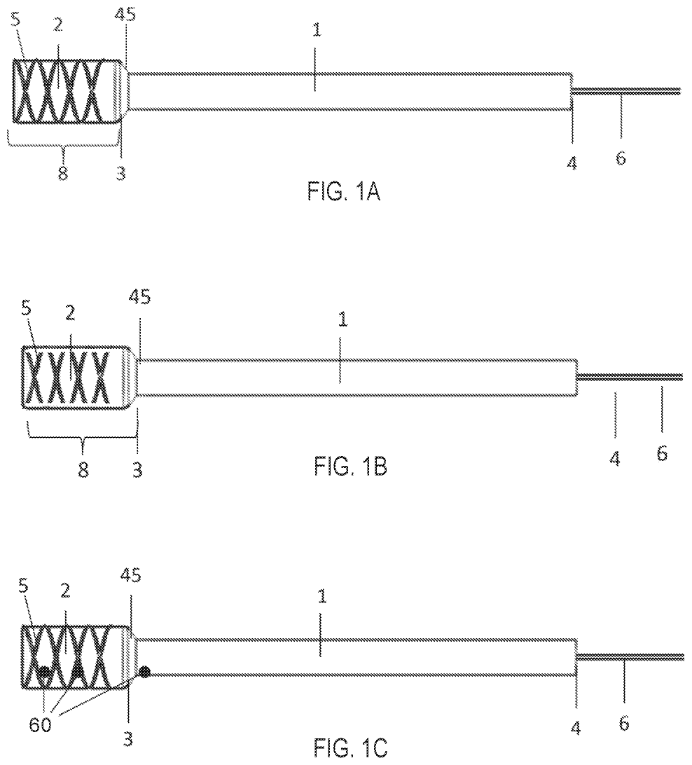

FIGS. 1A-1C are preferred embodiments of a device that may be used for post-operative wound treatment with an elongate shaft 1, wound dressing 2, and removal element 5. FIGS. 1A-1C each illustrate an embodiment with optional features, any of which may be used or substituted with other features in other embodiments discussed herein. In particular, FIG. 1A shows a side view of an exemplary embodiment of a device for post-operative wound treatment having a removal element that encompasses a sponge, in accordance with some embodiments. Additionally, FIG. 1B shows a side view of an exemplary embodiment of a device for post-operative wound treatment having a removal element that is woven through a sponge, in accordance with some embodiments. Further, FIG. 1C shows a side view of an exemplary embodiment of a device for post-operative wound treatment having radiopaque markers 60 on components of the device, in accordance with some embodiments.

As seen in FIGS. 1A-1C, the elongate shaft 1 may have an internal end 3 that is disposed in the patient's wound and an external end 4 that is disposed outside the patient. The elongate shaft 1 may be a tube, cannula, catheter, or other hollow structure preferably having a lumen extending the length thereof and that is sized to receive the wound dressing 2 and removal element 5 in a compressed configuration. As seen in FIGS. 1A and 1C, removal element 5 may be wrapped around wound dressing 2. As seen in FIG. 1B, removal element 5 may be woven through wound dressing 2. In some embodiments, wound dressing 2 may comprises a sponge. Further, as seen in FIG. 1C, elongate shaft 1, wound dressing 2, and removal element 5 may each comprise radiopaque markers.

The elongate shaft 1 may have a flared 45 internal end 3 to allow dressing guidance to be slidably advanced into the lumen during removal by proximal retraction of the removal element 5. The elongate shaft 1 may have a single lumen. Optionally, in any embodiment, the elongate shaft 1 may have multiple lumens in order to provide fluid suction and/or irrigation. For example, the elongate shaft 1 may have three lumens (one lumen for suction, one lumen for irrigation, and one lumen for device retraction) or the elongate shaft 1 may have three or more lumens (one or more lumens for suction, one or more lumens for irrigation, and one lumen for device retraction). The elongate shaft 1 preferably has a circular cross-section. Optionally, in any embodiment, the elongate shaft 1 may have various cross-sectional shapes known to those skilled in the art, such as an ovular cross-section or a rectangular cross-section. The elongate shaft 1 is preferably formed from a sterile flexible polymer. Any suitable material may be used including elastomeric or polymeric materials including, but not limited to, silicone or medical-grade polyvinyl chloride (PVC). The elongate shaft 1 is preferably formed from a uniform material. Optionally, in any embodiment, the elongate shaft 1 may have sections of varying durometer in order to control stiffness, flexibility or other mechanical properties of the elongate shaft 1. Optionally, in any embodiment, the elongate shaft 1 may be formed from multiple materials to provide desirable mechanical properties to the elongate shaft.

The wound dressing 2 is preferably a sterile, open cell reticulated, hydrophobic, polyurethane foam. The wound dressing 2 preferably has pore sizes of approximately 400 to 600 microns. Optionally, in any embodiment, the dressing 2 may have silver or antimicrobial agents added thereto. Optionally, in any embodiment, the dressing 2 may be non-open cell reticulated foam. Optionally, in any embodiment, the dressing 2 may be polyvinyl alcohol foam or any other material. Optionally, in any embodiment, the dressing 2 may have pore sizes of approximately 60 to 400 microns. Optionally, in any embodiment, the dressing 2 may have pore sizes that vary along the length of the dressing 2. Optionally, in any embodiment, the dressing 2 may have pore sizes that vary along the circumference of the dressing 2. Optionally, in any embodiment, the dressing 2 may have pore sizes that vary along any other direction. Optionally, in any embodiment, the dressing 2 may have multiple stacked layers with different materials to change the suction and dispersion properties of the dressing 2. For example, the dressing 2 may have a silver-coated polyurethane layer, a polyvinyl alcohol foam layer, and a waterproof adhesive layer. The stacked layers may be the same thickness or have varying thicknesses. The dressing 2 sizes may have surface areas up to 1000-1500 cm². The dressing 2 may be cylindrically shaped. Optionally, in any embodiment, the dressing 2 may be rectangular or another shape. The dressing 2 may have a long side (i.e. long length) and two short sides (i e small width and height). The dressing 2 may be collapsed with a narrowing of the width and height. Optionally, in any embodiment, the dressing 2 may be flexible so that it may be manipulated. The dressing 2 may be fabricated to allow equal distribution of negative pressure across the wound. The dressing 2 materials and configurations will be apparent to those skilled in the art and may vary depending on factors including patient's anatomy, exudate quantity, and/or state of the wound.

The removal element 5 may be a type of helical braid woven such that as the braid lengthens, it narrows. The removal element 5 may have a distal end 8 that is configured to be disposed in the wound and a trailing end 6 that extends proximally from the wound toward an external surface of the patient. The removal element 5 may be collapsible. The distal end 8 may be of diameter or width greater than the lumen of the elongate shaft 1 in its expanded state. The distal end 8 may be of diameter or width less than the lumen of the elongate shaft 1 in its collapsed state. Optionally, in any embodiment, the distal end 8 may conform to the wound dressing 2 contour. Optionally, in any embodiment, the distal end 8 may be bulbous, cylindrical, rectangular, or any other shape. The components of the removal element 5 may come together on one end of the distal end 8 to create a trailing end 6 which forms a tether that may be retracted proximally by a physician or other operator when removal is desired. The components may come together in a knot, be bonded together, or any other known processes of joining. The trailing end 6 may be a tight braid or bonded components that extend from distal end 8. Optionally, in any embodiment, the trailing end 6 may be a continuation of the distal end 8 helical braid.

The removal element 5 preferably encompasses the dressing 2 with a tight fit such that the dressing is entrapped by the removal element. Optionally, in any embodiment, the removal element 5 may be woven into the periphery of the dressing 2. Optionally, in any embodiment, the removal element 5 may be woven through the center of the dressing 2. Optionally, in any embodiment, the removal element 5 may be woven around the dressing 2, woven into the periphery of the dressing, and/or woven through the center of the dressing 2, or any combination thereof. The long-side of the dressing 2 may be aligned or substantially parallel with the removal element 5 longitudinal axis. The trailing end 6 of the removal element 5 may span the length of the elongate shaft 1 from the internal end 3, through the external end 4 with an additional length extending therepast that may be grasped by an operator or coupled to an actuation mechanism for retraction. The trailing end 6 may be pulled taut so that the removal element 5 captures the dressing 2 and also so that the internal end 3 of the elongate shaft 1 abuts the wound dressing 2. Optionally, in any embodiment, the elongate shaft 1 may be connected to the wound dressing 2 with adhesive or other method of bonding.

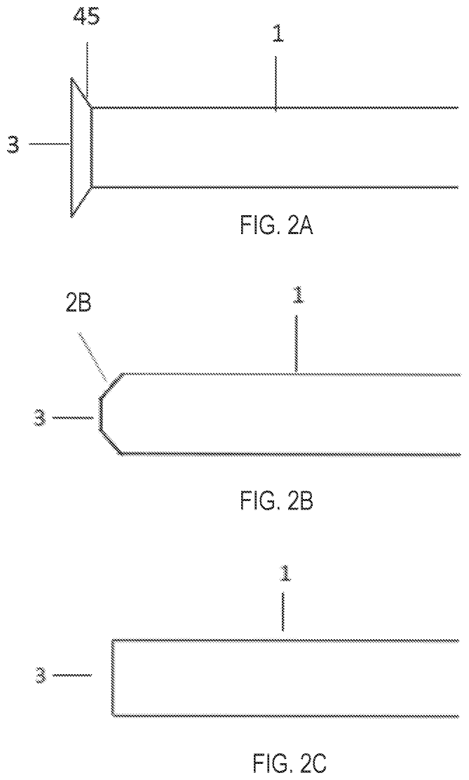

FIG. 2A-2C are preferred embodiments of the elongate shaft 1, as depicted in FIGS. 1A, 1B, and/or 1C and may be used in this or any other embodiment of the device. In particular, FIG. 2A shows a side view of an exemplary embodiment of the internal end of the elongate shaft, the internal end having a flare in accordance with some embodiments. Additionally, FIG. 2B shows a side view of an exemplary embodiment of the internal end of the elongate shaft, the internal end having an external chamfer 61 in accordance with some embodiments. Further, FIG. 2C shows a side view of an exemplary embodiment of the internal end of the elongate shaft, the internal end having no flare in accordance with some embodiments.

The elongate shaft 1 preferably has a length of 0.5 m to 3 m. More preferably, the elongate shaft 1 has a length of 1 m to 2 m. The elongate shaft 1 preferably has an inner diameter of 2 mm to 10 mm More preferably, the elongate shaft 1 has an inner diameter of 3 mm to 5 mm. The elongate shaft 1 preferably has an outer diameter of 4 mm to 15 mm More preferably, the elongate shaft 1 has an outer diameter of 5 mm to 7 mm. The elongate shaft 1 may have a flared 45 internal end 3 to allow dressing guidance into the lumen during removal by proximal retraction of the dressing into the lumen of the elongate shaft 1. The flared diameter 45 is preferably 5 mm to 20 mm More preferably, the flared diameter 45 is 7 mm to 15 mm Optionally, in any embodiment and as illustrated in FIG. 2A, the flared 45 internal end 3 may be flexible so that it easily collapses as it is pulled through the wound during removal. Optionally, in any embodiment, the internal end 3 may be the same diameter as the rest of the elongate shaft 1. Optionally, in any embodiment and as illustrated in FIG. 2B, end 3 may have an external chamfer 2B. Optionally, in any embodiment and as illustrated in FIG. 2C, end 3 may have no flare.

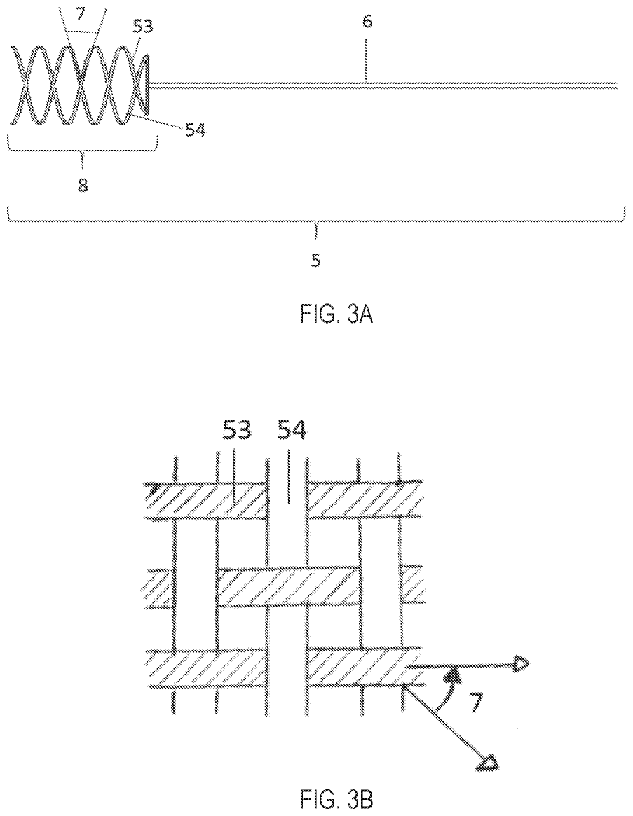

FIG. 3A is a preferred embodiment of the removal element 5, as depicted in FIGS. 1A, 1B, and/or 1C and may be used in this or any other embodiment of the device. The removal element 5 may be a type of helical braid woven such that as the braid lengthens, it narrows due to the reduced angle 7 between the warp 53 and weft 54 components at their crossing points. The removal element 5 may be composed of any type of fiber, wire, or tube. Optionally, in any embodiment, the removal element 5 may be a cage, expandable member, or take any other form. The removal element 5 is preferably a biaxial braid. FIG. 3B shows a flat-version of a biaxial braid, as a preferred embodiment of the removal element shown in FIG. 3A. The removal element 5 may be composed of a plurality of intertwining components. The removal element 5 preferably is composed of between 4 and 16 intertwining components. More preferably, the removal element 5 is composed of 4 to 8 intertwining components. The fibers may be hollow. The removal element 5 may be woven in any pattern. The removal element 5 is preferably woven with pairs of warp 53 and weft 54 components that alternatively pass over and under each other, with one pair spiraling clockwise and the other pair spiraling counter-clockwise. The removal element 5 preferably has a 30 to 60-degree angle 7 between the warp 53 and weft 54 components. More preferably, the angle 7 between the warp 53 and weft 54 components is 40 to 50-degrees. The intertwining components are preferably flat with widths of 0.1 mm to 1 mm. More preferably, the intertwining components have widths of 0.1 mm to 0.3 mm Optionally, in any embodiment, the removal element 5 may be multi-directional or unidirectional braids. Optionally, in any embodiment, the removal element 5 may have lower angle 7 orientations to change the expansion and compression properties of the removal element. Optionally, in any embodiment, the removal element 5 may have higher angle 7 orientations to change the expansion and compression properties of the removal element. The removal element 5 may be self-expanding. The removal element 5 may be sterile polypropylene fiber or any suitable fiber. The removal element 5 may be a flexible wire. The removal element 5 may have a trailing end 6 at one end that is of smaller diameter or width than the elongate shaft 1 (shown in FIGS. 1A, 1B, and/or 1C). The distal end 8 may be woven to conform to the wound dressing 2 contour. The distal end 8 may have a bulbous shape that is slightly larger than the corresponding wound dressing 2. The distal end 8 may have a cylindrical shape or any other shape. The distal end 8 may be larger in diameter or width than the lumen of the elongate shaft 1 (shown in FIGS. 1A, 1B, and/or 1C) in its expanded shape. The distal end 8 may be collapsible to a diameter or width smaller than the lumen of the elongate shaft 1 (shown in FIGS. 1A, 1B, and/or 1C). The intertwining components of the removal element 5 may come together on a distal portion of the distal end 8 to create a trailing end 6 that acts as a tether so the removal element may be pulled proximally The intertwining components may come together in a knot, be bonded together, or any other known processes of joining, or they may be twisted or braided together to form a single tether. The trailing end 6 may be a tight braid or bonded components that extend proximally from the distal end 8. The trailing end 6 may have a circular cross-section. Optionally, in any embodiment, the trailing end 6 may have a rectangular cross-section forming a flat ribbon or any other shape. The trailing end 6 preferably has a diameter or maximum width of 0.1 mm to 1.2 mm More preferably, the diameter or maximum width is 0.2 mm to 0.8 mm. The removal element 5 may be woven so that as the trailing end 6 is pulled axially, the circumference of the distal end 8 narrows and the distal end 8 collapses to engage and capture the dressing 2 (not pictured) so that as the removal element is retracted proximally it will also carry the dressing 2 with it in the proximal direction.

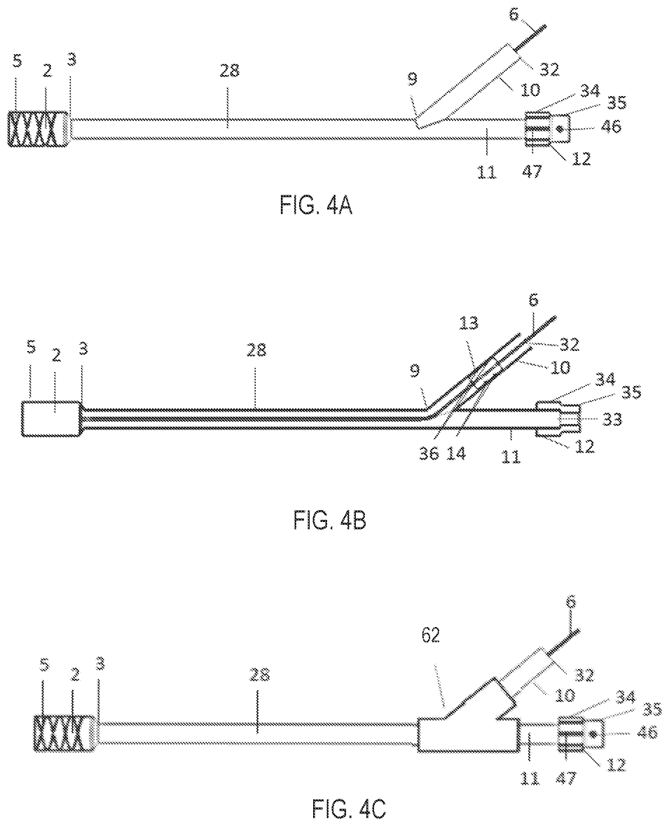

FIGS. 4A and 4B and 4C each illustrate a preferred embodiment of a device that may be used for post-operative wound treatment with elongate shaft 28, wound dressing 2, removal element 5, external fitting 12, and stopper 13. FIG. 4A shows a side view of the embodiment and FIG. 4B shows a cross-section view of the embodiment. Additionally, FIG. 4C shows a side view of the embodiment having a y-fitting 62. FIGS. 4A and 4B and 4C each illustrate an embodiment with optional features, any of which may be used or substituted with other features in other embodiments discussed herein.

The elongate shaft 28 may have an internal end 3 that is disposed in the wound in the patient and a y-split 9 diverging into two external arms 10, 11 with two external ends 32, 33 preferably disposed outside of the wound and externally to the patient. Optionally, in any embodiment, the y-split 9 may be a t-split or any other divergence of one member into two. For example, the first external arm 11 may extend in a direction substantially parallel with the internal tubing axis and the second external arm 10 may diverge at a 20 to 60-degree angle. More preferably, the second external arm 10 diverges at a 30 to 40-degree angle. The elongate shaft 28 is preferably formed from a sterile flexible polymer. Any suitable material may be used including elastomeric or polymeric materials including, but not limited to, silicone or medical-grade polyvinyl chloride (PVC). The elongate shaft 28 preferably has an approximate length of 0.5 m to 3 m (from internal end to each external end). More preferably, the elongate shaft 28 has a length of 1 m to 2 m. Optionally, in any embodiment, the elongate shaft 28 may have any length to accommodate varying patient anatomy and use. The elongate shaft 28 preferably has an inner diameter of 2 mm to 10 mm More preferably, the elongate shaft 28 has an inner diameter of 3 mm to 5 mm. The elongate shaft 28 preferably has an outer diameter of 4 mm to 15 mm More preferably, the elongate shaft 28 has an outer diameter of 5 mm to 7 mm. The y-split 9 may allow the trailing end 6 of the removal element 5 to exit the external end 32 of the tubing without interfering with the connection to the external device for suction and irrigation.

The wound dressing 2 and removal element 5 may be the same as those described in FIGS. 1A, 1B, and/or 1C.

The fitting 12 may be any medical tubing standard fitting known in the art such as a cylindrical shape. The fitting 12 may be configured to mate with commercial suction and/or irrigation device tubing. The fitting 12 outer diameter may be stepped. The fitting 12 preferably has a first outer diameter 34 of approximately 8 mm to 17 mm More preferably, the fitting 12 has a first outer diameter 34 of 10 mm to 15 mm. The outer diameter 34 may have surface features such as ridges 47 around the circumference to help an operator grasp the fitting. The ridges 47 may have a square shape. Optionally, in any embodiment, the ridges 47 may be any shape. The ridges 47 preferably have widths of approximately 0.1 mm to 1 mm More preferably, the ridges 47 have widths of 0.2 mm to 0.5 mm. The fitting 12 preferably has a second outer diameter 35 of approximately 5 mm to 13 mm More preferably, the fitting 12 has a second outer diameter 35 of 7 mm to 10 mm. The second outer diameter 15 may have a locking pin 46 so that an external device female fitting can slide into the fitting 12 with the locking pin 46 entering a channel on the external fitting such that as the fitting is rotated, the locking pin 46 locks to the fitting. Optionally, in any embodiment, the outer diameter 35 may have any locking feature such as a threaded region as used in a Luer lock. The fitting 12 preferably has an inner diameter of 4 mm to 8 mm More preferably, the fitting 12 has an inner diameter of 5 mm to 7 mm. The fitting 12 is preferably sterile acrylonitrile butadiene styrene (ABS) or another suitable polymer. Optionally, in any embodiment, the fitting 12 may be any suitable metal, ceramic, or other material.

A stopper 13 is preferably disposed in the opposite arm 10 of the y-split 9. The stopper 13 may be of cylindrical shape or any other shape. The stopper 13 preferably has a diameter of approximately 2 mm to 6 mm More preferably, the stopper 13 has a diameter of 2.5 mm to 4.5 mm. The stopper 13 preferably has a length of 5 mm to 25 mm More preferably, the stopper 13 has a length of 10 mm to 20 mm. The stopper 13 body is preferably sterile acrylonitrile butadiene styrene (ABS) or a comparable suitable polymer. Optionally, in any embodiment, the stopper 13 body may be any suitable metal, ceramic or other material. The stopper 13 preferably has multiple circumferential grooves 36 disposed therearound and sized to fit an external O-ring 14 in each. The circumferential grooves 36 may have rectangular cross-sections. Optionally, in any embodiment, the circumferential grooves 36 may be semi-circles or any shape. Optionally, in any embodiment, the stopper 13 may have other mechanical features to capture an O-ring 14. Optionally, in any embodiment, the O-rings 14 may be bonded to the stopper 13. The O-rings 14 preferably have outer diameters of approximately 2 mm to 6 mm More preferably, the O-rings 14 have outer diameters of 3.1 mm to 5.1 mm. The O-rings 14 preferably have circular cross-sections. Optionally, in any embodiment, the O-rings 14 may have any shape cross-section. The O-rings 14 may have a press fit with the inner lumen of the arm 10 of the elongate shaft 28 to provide a seal. The O-rings 14 are preferably sterile rubber. Optionally, in any embodiment, the O-rings 14 may be any suitable elastomer.

The removal element 5 preferably encompasses the dressing 2 with a tight fit such that the dressing is entrapped by the removal element. The long-side of the dressing 2 may be aligned or substantially parallel with the removal element 5 longitudinal axis. The first external end 33 has a fitting 12 to connect to a suction and/or irrigation device canister tubing. The fitting 12 may be bonded to the external end 33 of the elongate shaft 28 using an adhesive. Optionally, in any embodiment, the fitting 12 may be connected to the external end 33 of the elongate shaft 28 by any bonding method including but not limited to mechanical bonding, chemical bonding, adhesives, ultrasound welding, solvent bonding, etc. The trailing end 6 of the removal element 5 spans the length of the lumen of the elongate shaft 28 from the internal end 3, through the second external arm 10, exiting through the second external end 32, disposed external to the patient. The trailing end 6 is pulled taut so that the removal element 5 captures the dressing 2 and also so that the internal end 3 of the elongate shaft 3 abuts the wound dressing 2. Optionally, in any embodiment, the elongate shaft 28 may be connected to the wound dressing 2 with adhesive or other method of bonding. A stopper 13 captures the trailing end 6 of the removal element 5 in the second external arm 10 of the elongate shaft 28 such that as the trailing end 6 is pulled axially, the stopper 13 also moves axially, allowing for removal element 5 collapse and subsequent collapse of the dressing 2 for device removal. The trailing end 6 may be captured by the stopper 13 during a molding process. Optionally, in any embodiment, the trailing end 6 may be captured by the stopper 13 by mechanical mechanisms or bonding. The stopper 13 may maintain the seal within the elongate shaft 28 through a press fit between the O-rings 17 and the inner lumen of the elongate shaft 28.

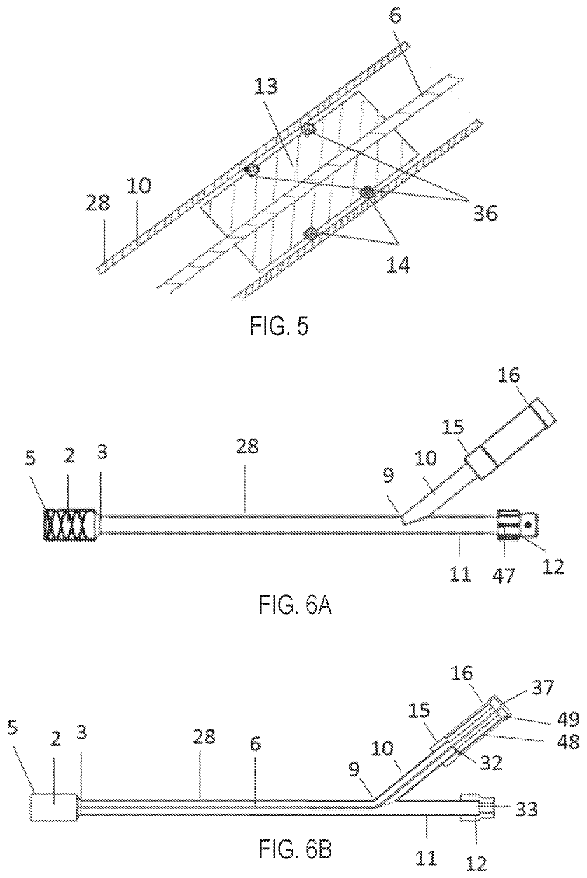

FIG. 5 is a cross-section view of the stopper 13 as described in FIGS. 4A and 4B with multiple circumferential grooves 36 and multiple O-rings 14 to provide a press fit with the second external arm 10 of the elongate shaft 28.

FIGS. 6A and 6B each illustrate a preferred embodiment of a device that may be used for post-operative wound treatment with elongate shaft 28, wound dressing 2, removal element 5, external fitting 12, threaded fitting 15, and threaded cap 16. FIG. 6A shows a side-view of the embodiment and FIG. 6B shows a cross-sectioned view of the embodiment. FIGS. 6A and 6B each show the elongate shaft 28 with y-split 9 and external fitting 12 on one external arm 11 as described in FIGS. 4A, 4B and/or 4C and the wound dressing 2 and removal element 5 as described in FIGS. 1A, 1B, and/or 1C. FIGS. 6A and 6B each illustrate an embodiment with optional features, any of which may be used or substituted with other features in other embodiments discussed herein.

The externally threaded fitting 15 is preferably of cylindrical shape. The fitting 15 preferably has an inner diameter of approximately 4 mm to 8 mm More preferably, the fitting 15 has an inner diameter of 5 mm to 7 mm. The fitting 15 preferably has an external diameter of 6 mm to 10 mm More preferably, the fitting 15 has an external diameter of 7 mm to 9 mm. The internally threaded cap 16 is preferably of cylindrical shape. Optionally, in any embodiment, the cap 16 may be any shape. The cap 16 preferably has a cylindrical lumen sized to mate with the externally threaded fitting 15. The fitting 15 preferably has external threads 48 along a portion of the length of the tubular body. Optionally, in any embodiment, the fitting 15 may be threaded along its entire length. The cap 16 preferably has internal threads 49 along a portion of the length of the tubular body. Optionally, in any embodiment, the cap 16 may be threaded along its entire length. The fitting 15 length is preferably 5 mm to 100 mm More preferably, the fitting 15 length is 5 mm to 20 mm. The cap 16 length is preferably 5 mm to 100 mm More preferably, the cap 16 length is 5 mm to 20 mm. The trailing end 6 of the removal element 5 may be attached to the inner end 37 of the cap 16 by being molded into it. Optionally, in any embodiment, the trailing end 6 may be attached to the cap 16 by any mechanical mechanism or bonding. The fitting 15 is preferably acrylonitrile butadiene styrene (ABS). Optionally, in any embodiment, the fitting 15 is any suitable polymer. The cap 16 is preferably acrylonitrile butadiene styrene (ABS). Optionally, in any embodiment, the cap 16 is any suitable polymer.

The removal element 5 preferably encompasses the dressing 2 with a tight fit such that the dressing is entrapped by the removal element. The long-side of the dressing 2 may be aligned or substantially parallel with the removal element 5 longitudinal axis. The first external end 33 may have a fitting 12 to connect to suction and/or irrigation device canister tubing. The fitting 12 may be bonded to the external end 33 of the elongate shaft 28 using adhesive. Optionally, in any embodiment, the fitting 12 may be connected to the external end 33 of the elongate shaft 28 by any bonding method. The second external end 32 may have an externally threaded fitting 15. The fitting 15 may be bonded to the second external end 32 using adhesive. Optionally, in any embodiment, the fitting 15 may be bonded to the second external end 32 by any bonding method. The trailing end 6 of the removal element 5 may span the length of the lumen of the elongate shaft 28 from the internal end 3, through the second external arm 10, through the second external end 32, to attach to the internally threaded cap 16. As the cap 16 is unscrewed from the fitting 15, the trailing end 6 of the removal element 5 is pulled axially. The trailing end 6 may be pulled taut by unthreading the cap 16 so that the removal element 5 captures the dressing 2 and also so that the internal end 3 of the elongate shaft 28 abuts the wound dressing 2. Optionally, in any embodiment, the elongate shaft 28 may be connected to the wound dressing 2 with adhesive or other method of bonding. The fitting 15 and cap 16 may have mating threads 48, 49 so that when engaged, a seal is maintained within the elongate shaft 28.

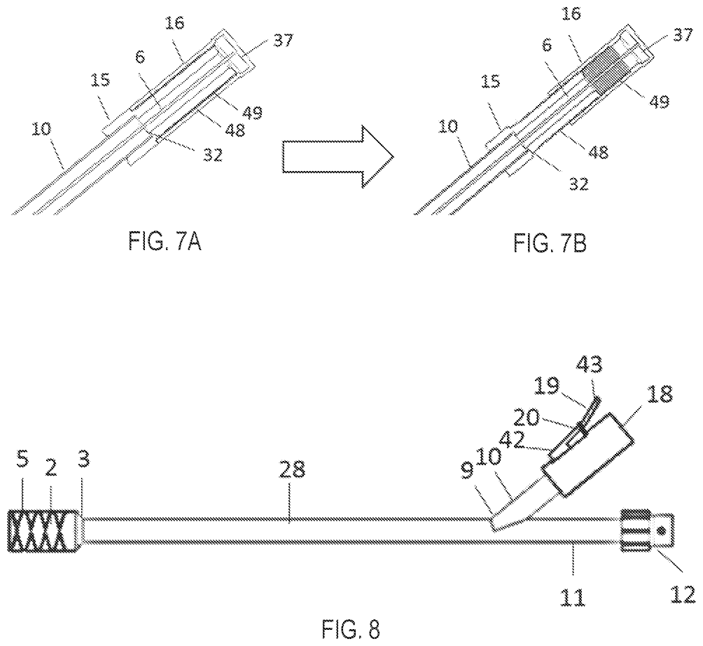

FIGS. 7A and/or 7B depicts a preferred embodiment of the externally threaded fitting 15 and the internally threaded cap 16, as described in FIGS. 6A and 6B. The fitting 15 attaches to the end 32 of one external arm 10. The fitting 15 and cap 16 may have mating threads 48, 49. The trailing end 6 of the removal element (not fully pictured) may attach to the inner end 37 of the cap 16. As the cap 16 is unscrewed from the fitting 15, the trailing end 6 of the removal element is pulled axially thereby pulling the dressing (not pictured) out of the wound into the elongate shaft 28.

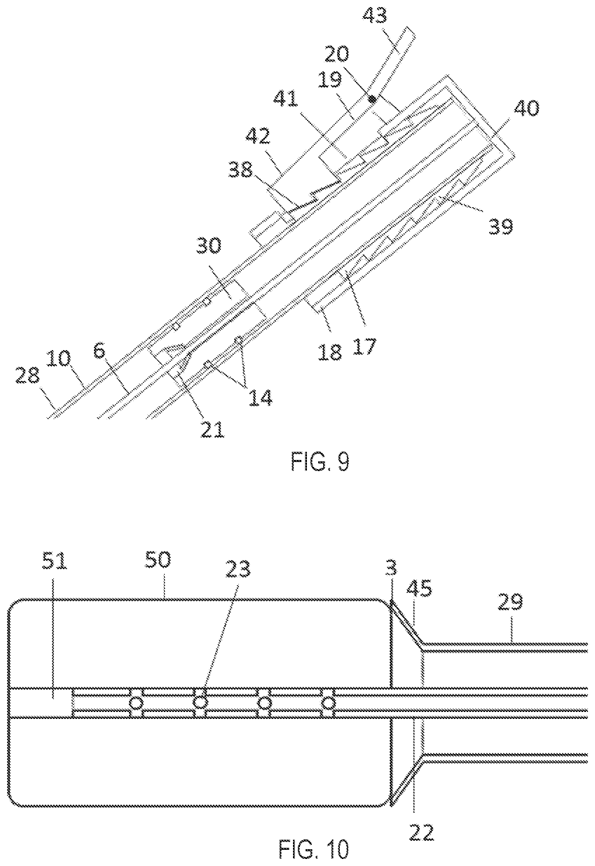

FIG. 8 is a preferred embodiment of a device that may be used for post-operative wound treatment with elongate shaft 28, wound dressing 2, removal element 5, external fitting 12, ratcheted fitting 17 (see e.g. FIG. 9), and cap 18. FIG. 8 shows the elongate shaft 28 with y-split 9 and external fitting 12 on one external arm 11 as described in FIGS. 4A, 4B, and/or 4C and the wound dressing 2 and removal element 5 as described in FIGS. 1A, 1B, and/or 1C. FIGS. 6A, 6B, and/or 8 illustrate embodiments with optional features, any of which may be used or substituted with other features in other embodiments discussed herein.

The ratcheted fitting 17 may be bonded to one external arm 10 of the elongate shaft 28. The ratcheted fitting 17 is preferably of cylindrical shape. Optionally, in any embodiment, the ratcheted fitting 17 may be any shape. The fitting 17 may have an inner diameter of approximately 5 to 7 mm. The fitting 17 preferably has ratchets 39 along a portion of the length of the tubular body. Optionally, in any embodiment, the fitting 17 may have ratchets (not pictured) along its entire length. The ratchet fitting 17 length is preferably 5 mm to 100 mm More preferably, the fitting 17 length is 5 mm to 20 mm. The ratchet cap 18 is preferably of cylindrical shape. Optionally, in any embodiment, the ratchet cap 18 may be any shape. The cap 18 preferably has an inner diameter that provides for a sliding fit with the fitting 17. The cap 18 length is preferably 5 mm to 100 mm More preferably, the cap 18 length is 5 mm to 20 mm. The ratchet cap 18 preferably has an actuatable arm 19 that is rotated about a pin 20. Optionally, in any embodiment, the cap 18 may also have an actuation mechanism 19 that moves by any mechanism such as a button, a lever, or any other mechanism. The actuation mechanism 19 may be an elongate rectangular shape with two ends 42, 43 on opposite sides of the pin 20. Optionally, in any embodiment, the actuation mechanism 19 may be any shape. The trailing end 6 of the removal element 5 may be attached to the inner end 40 of the cap 18 by being molded into it. Optionally, in any embodiment, the trailing end 6 may be attached to the cap 18 by any mechanical mechanism or bonding. The fitting 17 is preferably sterile acrylonitrile butadiene styrene (ABS). Optionally, in any embodiment, the fitting 17 may be any suitable polymer. The cap 18 is preferably sterile acrylonitrile butadiene styrene (ABS). Optionally, in any embodiment, the cap 18 may be any suitable polymer. The cap 18 is able to move axially along the fitting 17 when the actuation mechanism 19 is disengaged, pulling the trailing end 6 of the biaxial braid 5 axially thereby pulling the dressing 2 out of the wound into the elongate shaft 28.

The removal element 5 preferably encompasses the dressing 2 with a tight fit such that the dressing is entrapped by the removal element. The long-side of the dressing 2 may be aligned or substantially parallel with the removal element 5 longitudinal axis. The first external end 33 may have a fitting 12 to connect to suction and/or irrigation device canister tubing. The fitting 12 may be bonded to the external arm 10 of the elongate shaft 28 using adhesive. Optionally, in any embodiment, the fitting 12 may be bonded by any bonding method. The second arm 10 has a ratcheted fitting 17. The fitting 17 may be bonded using adhesive. Optionally, in any embodiment, the fitting 17 may be bonded by any bonding method. The trailing end (not pictured) of the removal element 5 may span the length of the lumen of the elongate shaft 28 from the internal end 3, through the second external arm 10, through the second external end (not pictured) disposed external to the patient, to attach to the ratchet cap 18. The trailing end may be pulled taut so that the removal element 5 captures the dressing 2 and also so that the removal element 5 captures the dressing 2 and also so that the internal end 3 of the elongate shaft 28 abuts the wound dressing 2. Optionally, in any embodiment, the elongate shaft 28 may be connected to the wound dressing 2 with adhesive or other method of bonding. The cap 18 actuation mechanism 19 may disengage, enabling the cap 18 to move axially, pulling the trailing end of the biaxial braid 5 axially thereby pulling the dressing 2 out of the wound into the elongate shaft 28.

FIG. 9 is a preferred embodiment of the ratcheted fitting 17 and cap 18 from FIG. 8. The cap 18 may have a cutout 41 that the actuation mechanism 19 can rotate into, about pin 20. The actuation mechanism 19 may have one or more ratchets 38 on one end 42. The actuation mechanism 19 may have ratchets 38 on one end 42. The ratchets 38 may be shaped to engage with the fitting ratchets 39. The actuation mechanism 19 ratchets 38 and fitting ratchets 39 may be a triangular shape or form steps. The pitch of the ratchets 38, 39 may be adjusted to any desired spacing in order to control coarseness or fineness of adjustment. Optionally, in any embodiment, the actuation mechanism 19 ratchets 38 and fitting ratchets 39 may be any mating shape. The cap 18 is able to move axially along the fitting 17 when the actuation mechanism 19 is disengaged by pushing on one end 43, pulling the trailing end 6 of the biaxial braid (not fully pictured) axially thereby pulling the dressing (not pictured) out of the wound into the elongate shaft 28.

Optionally in any embodiment, the external ratchets 39 may be molded as part of the external surface of the second external arm 10 of the elongate shaft 28.

Optionally in any embodiment, there may be a stopper 30 in the inner lumen of the second external arm 10 of the elongate shaft 28, as described in FIGS. 4A, 4B, and/or 4C. The stopper 30 maintains the pressure seal in the elongate shaft 28.

Optionally in any embodiment, the stopper 30 may also have a cone-shaped custom O-ring 21. The O-ring 21 preferably has an inner diameter of 0.1 to 0.8 mm. The O-ring 21 may be captured in the stopper 30 by bonding or adhesive. Optionally, in any embodiment, the O-ring 21 may be captured by any mechanical feature. The axis of the O-ring 21 may be aligned with the center axis of the stopper 30. The trailing end 6 of the removal element (not fully pictured) may be able to pass through the center axis of the stopper 30 and custom O-ring 21 while maintaining the seal with a tight fit between the trailing end 6 and the O-ring 21.

The cap 18 actuation mechanism 19 may disengage to cause the cap ratchets 38 to release the fitting ratchets 39, enabling the cap 18 to move axially, pulling the trailing end 6 of the biaxial braid (not pictured) axially thereby pulling the dressing (not pictured) out of the wound into the elongate shaft 28.

FIG. 10 is a preferred embodiment of the internal end 3 of a multi lumen elongate shaft 29 and a cannulated wound dressing 50 with the removal element removed for convenient illustrative purposes although it is appreciated that the removal element is preferably included. The elongate shaft 29 may have two members, one that is connected to an inlet and one that is connected to an outlet. Each of the two members may be movable relative to one another. The two members may comprise a same material. The two members may comprise a similar material. The two members may be bonded together with adhesive. The two members may be bonded together using any known bonding process. The internal end 3 is flared to allow guidance into the lumen, as described in FIG. 2A.

The wound dressing 50 is preferably a sterile, open cell reticulated, hydrophobic, polyurethane foam. The wound dressing 50 preferably has pore sizes of approximately 400 to 600 microns. Optionally, in any embodiment, the dressing 50 may have silver or antimicrobial additions. Optionally, in any embodiment, the dressing 50 may be non-open cell reticulated foam. Optionally, in any embodiment, the dressing 50 may be polyvinyl alcohol foam. Optionally, in any embodiment, the dressing 50 may have pore sizes of approximately 60 to 400 microns. Optionally, in any embodiment, the dressing 50 may have multiple layers with different materials to change the suction and dispersion properties of the dressing 50. For example, the dressing 50 may have a silver-coated polyurethane layer, a polyvinyl alcohol foam layer, and a waterproof adhesive layer. The stacked layers may be the same thickness or have varying thicknesses. Dressing 50 sizes may have surface areas up to 1000-1500 cm². The dressing 50 may be cylindrically shaped. Optionally, in any embodiment, the dressing 50 may be rectangular or another shape. The dressing 50 may have a long side (i.e. long length) and two short sides (i e small width and height). The dressing 50 may be collapsed with a narrowing of the width and height. The dressing 50 may be cannulated through the center axis, along the long side, to allow for a hollow shaft to pass through. The cannula 51 may extend through the entire dressing 50. Optionally, in any embodiment, the cannula 51 may extend only partially through the dressing 50. Optionally, in any embodiment, the dressing 50 may be flexible so that it may be manipulated. The dressing 50 may be fabricated to allow equal distribution of negative pressure across the wound. The dressing 50 materials and configurations will be apparent to those skilled in the art and may vary depending on factors including patient's anatomy, exudate quantity, and/or state of the wound.

The elongate shaft 29 may have an internal end 3 that is disposed in the patient's wound and an external end (not pictured) that is disposed outside the patient. The elongate shaft 29 may be a tube, cannula, catheter, or other hollow structure that is sized to receive the wound dressing 50 and removal element in a compressed configuration. The elongate shaft 29 may have a flared 45 internal end 3 to allow dressing guidance to be slidably advanced into the lumen during removal by proximal retraction of the removal element 5. Optionally, in any embodiment, the elongate shaft 29 may have various cross-sectional shapes known to those skilled in the art, such as an ovular cross-section or a rectangular cross-section. The elongate shaft 29 is preferably formed from a sterile flexible polymer. Any suitable material may be used including elastomeric or polymeric materials including, but not limited to, silicone or medical-grade polyvinyl chloride (PVC). The elongate shaft 29 is preferably formed from a uniform material. Optionally, in any embodiment, the elongate shaft 29 may have sections of varying durometer in order to control stiffness, flexibility or other mechanical properties of the elongate shaft 29. Optionally, in any embodiment, the elongate shaft 29 may be formed from multiple materials to provide desirable mechanical properties to the elongate shaft.

A piece of tubing from one of the lumens from the elongate shaft 29 extends distally from the internal end 3 to form a hollow extension 22. The hollow extension 22 is preferably tubular with a diameter of 0.5 mm to 5 mm More preferably, the hollow extension 22 diameter is 1 mm to 3 mm Optionally, in any embodiment, the hollow extension 22 may have any shape. For example, the lumen of the elongate shaft 29 may be divided into two equal lumens down with the hollow extension 22 having a D-shaped half-circle cross-section. The hollow extension 22 may extend internally into the wound dressing 50. The hollow extension 22 may extend distally the length of the wound dressing 50. Optionally, in any embodiment, the hollow extension 22 may extend distally partially across the wound dressing 50. The hollow extension 22 may have an open proximal tip. Optionally, in any embodiment, the hollow extension 22 may have a closed tip. The hollow extension 22 may have a plurality of holes 23 distributed circumferentially. The hollow extension 22 may have a plurality of holes 23 distributed axially. Optionally, in any embodiment, the hollow extension 22 may have a plurality of holes 23 distributed circumferentially and axially in any pattern. The plurality of holes 23 may provide more distributed suctioning of exudates and/or distribution of irrigating fluids. The holes 23 preferably have a diameter of 0.1 mm to 1.5 mm More preferably, the holes 23 have a diameter of 0.2 mm to 0.8 mm in diameter. Optionally, in any embodiment, the holes 23 may be various sizes. The hollow extension 22 is preferably of the same material as the body of the elongate shaft 29. Optionally, in any embodiment, the hollow extension 22 is a different material than that of the elongate shaft 29. Optionally, in any embodiment, the hollow extension 22 is a different durometer than that of the elongate shaft 29.

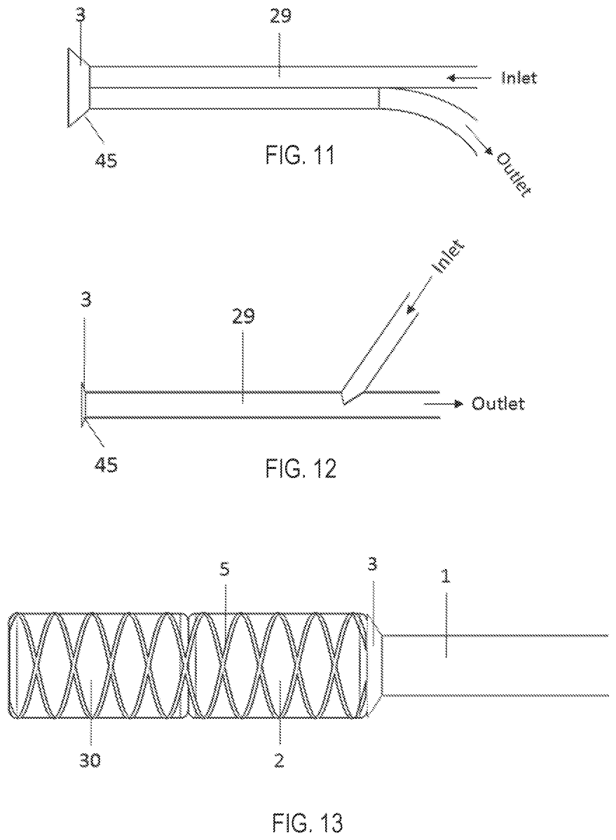

With reference to FIG. 11, in one embodiment, there may be two identical elongate shafts 29 that are bonded together along their length. The elongate shafts 29 may be bonded together with adhesive or any processes of bonding. The elongate shaft 29 may have a flared 45 internal end 3. The elongate shafts 29 may be a tube, cannula, catheter, or other hollow structure. The elongate shafts 29 may have a single lumen extending the length of the shaft. The elongate shafts 29 may have a circular cross-section. The elongate shafts 29 may be a sterile flexible polymer or other material. The identical elongate shafts 29 may be pulled apart for convenience while attaching to an external system for separate inlet and outlet channels, one for suction and one for irrigation. The removal element (not pictured) may span either channel. Alternatively, the construct of the two elongate shafts may be co-extruded to form a single integral component with two lumens/channels.

With reference to FIG. 12, in one embodiment, there is one elongate shaft 29 with a y-connection that allows for two end-connections to external system tubing, one for inlet and the other for outlet, one for suction and one for irrigation. The removal element (not pictured) may span either channel. The elongate shafts 29 may have a flared 45 internal end 3. The elongate shafts 29 may be a tube, cannula, catheter, or other hollow structure. The elongate shafts 29 may have a single lumen that span the length of the shaft. The elongate shafts 29 may have a circular cross-section. The elongate shafts 29 may be a sterile flexible polymer or any material.

FIG. 13 is a preferred embodiment of a series of wound dressings 2, 30 captured by the removal element 5 to fill a larger wound space. Optionally, in any embodiment, wound dressings 2, 30 in various configurations may be captured by the removal element 5. The elongate shaft 1 and removal element 5 may be the same as those previously described in FIGS. 1A, 1B, and/or 1C. The wound dressings 2, 30 may be a sterile, open cell reticulated, hydrophobic, polyurethane foam. The wound dressings 2, 30 may have pore sizes of approximately 400 to 600 microns. Optionally, in any embodiment, the dressings 2, 30 may be polyvinyl alcohol foam or any other material. Optionally, in any embodiment, the dressings 2, 30 may have pore sizes of approximately 60 to 400 microns. Optionally, in any embodiment, the dressings 2, 30 may have pore sizes that vary along the length of the dressings 2, 30. Optionally, in any embodiment, the dressings 2, 30 may have pore sizes that vary along the circumference of the dressings 2, 30. Optionally, in any embodiment, the dressings 2, 30 may have pore sizes that vary along any other direction. Optionally, in any embodiment, the dressings 2, 30 may have multiple stacked layers with different materials to change the suction and dispersion properties of the dressings 2, 30. For example, the dressings 2, 30 may have a silver-coated polyurethane layer, a polyvinyl alcohol foam layer, and a waterproof adhesive layer. The stacked layers may be the same thickness or have varying thicknesses. The dressings 2, 30 sizes may have surface areas up to 500-1500 cm². The dressings 2, 30 may be cylindrically shaped. Optionally, in any embodiment, the dressings 2, 30 may be rectangular or another shape. The dressings 2, 30 may have a long side (i.e. long length) and two short sides (i e small width and height). The dressings 2, 30 may be collapsed with a narrowing of the width and height. The dressings 2, 30 may be flexible so that they may be manipulated. The dressings 2, 30 may be fabricated to allow equal distribution of negative pressure across the wound. The removal element 5 may encompasses the dressings 2, 30 with a tight fit such that the dressing is entrapped by the removal element. The long-side of the dressings 2, 30 may be aligned or substantially parallel with the removal element 5 longitudinal axis. Operation of the device in FIG. 13 is generally the same as previously described in other embodiments. Retraction of the removal element 5 collapses the removal element 5 and captures the dressings 2, 30 which are then pulled into the internal end 3 of the elongate shaft 1 which can then be easily removed from the patient without requiring removal surgery.

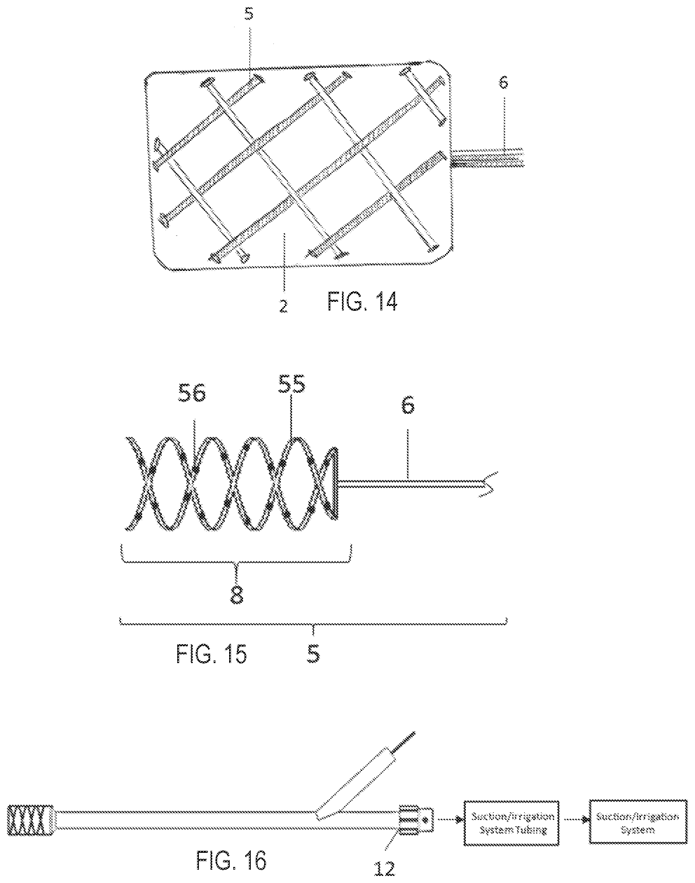

FIG. 14 is a preferred embodiment of the removal element 5 being intertwined into the wound dressing 2 with the elongate shaft removed for convenient illustrative purposes although it is appreciated that the elongate shaft is preferably included. The removal element 5 may be the same as that described in FIGS. 1A, 1B, and/or 1C. The removal element 5 may be a helical braid, such as a biaxial braid, with a trailing end 6. Optionally, in any embodiment, the removal element 5 may be woven into the periphery of the dressing 2. Optionally, in any embodiment, the removal element 5 may be woven through the center of the dressing 2. Optionally, in any embodiment, the removal element 5 may be woven around the dressing 2, woven into the periphery of the dressing, and/or woven through the center of the dressing 2, or any combination thereof.

FIG. 15 is a preferred embodiment of the removal element 5, as shown in FIGS. 3A and 3B, composed of hollow components 55. The hollow components 55 may be tubular or any shape. The hollow components 55 may be any type of fiber, metal, or other material. The hollow components 55 may be flexible. The hollow components 55 may have a plurality of ports 56 located along the length of the component at the distal end 8. The ports 56 may be holes. The ports 56 may be mesh. The ports 56 may be any other form that allows for fluid diffusion. Antibiotics may be distilled from the trailing end 6 (proximal end), to the distal end 8, exiting the plurality of ports 56 for even distribution in the wound.

FIG. 16 is a schematic of a preferred embodiment of the device, as shown in FIGS. 4A, 4B, and/or 4C, being used with suction and/or irrigation therapy. The device fitting 12 (as described in FIGS. 4A, 4B, and/or 4C) may connect to the suction and/or irrigation therapy system tubing. The suction and/or irrigation therapy may be an NPWT system. The suction therapy may be delivered from a portable or central suction system. The suction and/or irrigation may be provided from any system that is able to provide suction and/or irrigation through an elongate hollow shaft.

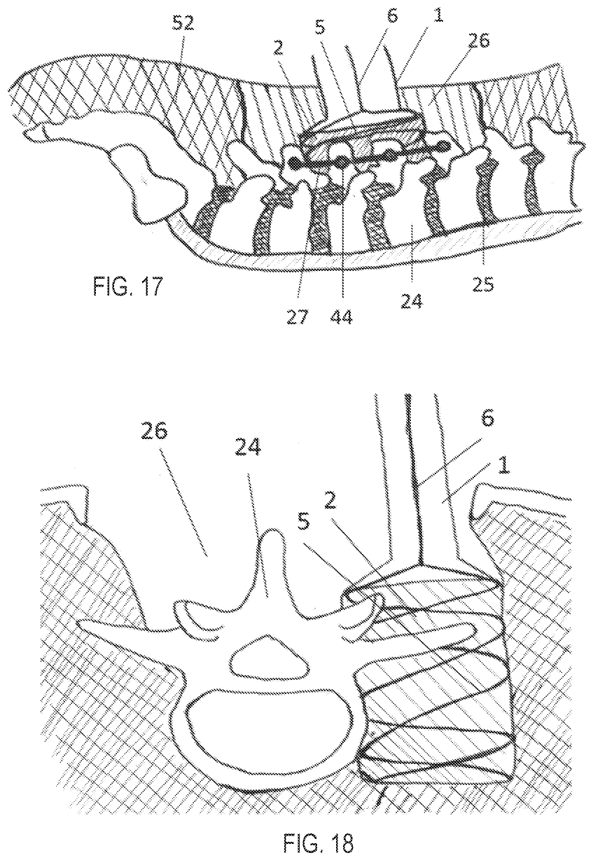

FIG. 17 shows a lateral view of the spine with vertebrae 24, disc space 25, and tissue 52. The device from FIGS. 4A, 4B, and/or 4C, including an elongate shaft 1, removal element 5 with trailing end 6, and wound dressing 2, is placed in the wound space 26 next to existing instrumentation, including rods 27 and screws 44. This device may be used with a suction and/or irrigation system or any of the other therapies described in this specification.

FIG. 18 shows a superior view of the spine with vertebra 24. The device from FIGS. 4A, 4B, and/or 4C, including an elongate shaft 1, removal element 5 with trailing end 6, and wound dressing 2, is placed in the wound space 26.

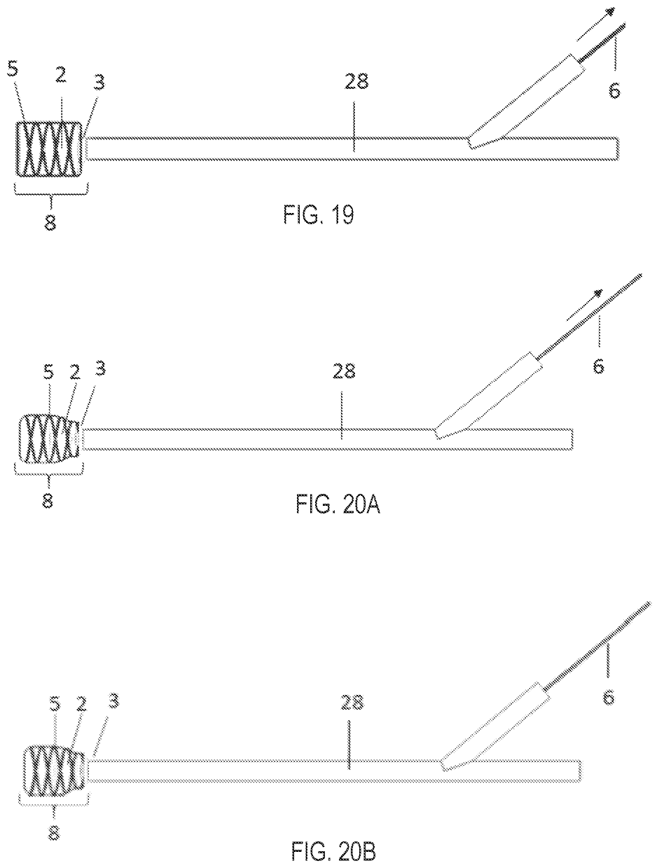

FIGS. 19-22 illustrate a method for device removal. In particular, FIG. 19 shows the trailing end 6 of the removal element 5 being pulled axially (in the direction of the arrow). The trailing end 6 of the removal element 5 spans the length of the lumen of the elongate shaft 28. The distal end 8 of the removal element 5 encompasses the wound dressing 2. The elongate shaft 28 internal end 3 abuts the wound dressing 2. FIG. 19 shows the device as described in FIGS. 4A, 4B, and/or 4C.

FIG. 20A shows the distal end 8 of the removal element 5 narrowing, collapsing the wound dressing 2. The trailing end 6 of the removal element 5 spans the length of the lumen of the elongate shaft 28. The distal end 8 of the removal element 5 encompasses the wound dressing 2. The elongate shaft 28 internal end 3 abuts the wound dressing 2. FIG. 20A shows the device as described in FIGS. 4A, 4B, and/or 4C.

FIG. 20B shows a device having a partially retracted sponge in an initial position. FIG. 20B shows the device as described in FIGS. 4A, 4B, and/or 4C.

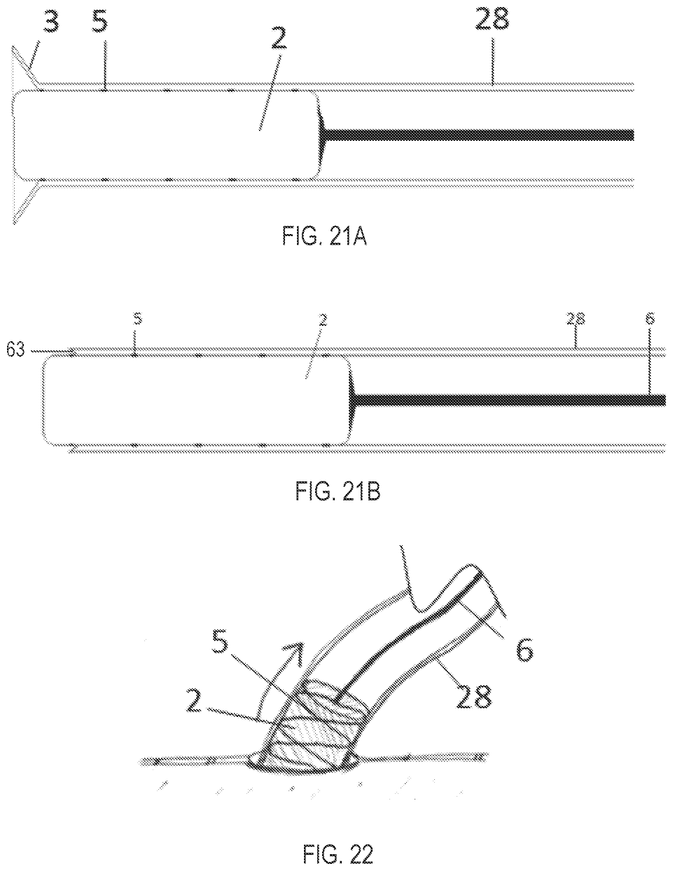

FIG. 21A shows the fully collapsed removal element 5 and wound dressing 2 after having been collapsed and retracted into the elongate shaft 28, proximally past the internal end 3 having a flare. FIG. 21A shows the device as described in FIGS. 4A, 4B, and/or 4C.

FIG. 21B shows the fully collapsed removal element 5 and wound dressing 2 after having been collapsed and retracted into the elongate shaft 28, proximally past the internal end 3 having an internal chamfer 63. FIG. 21B shows the device as described in FIGS. 4A, 4B, and/or 4C.

FIG. 22 shows the elongate shaft 28 being removed through the closed wound. The removal element 5 is fully collapsed around the wound dressing 2 and has been retracted into the elongate shaft 28. The trailing end 6 of the biaxial braid spans the length of the lumen of the elongate shaft 28. FIG. 22 shows the device as described in FIGS. 4A, 4B, and/or 4C.

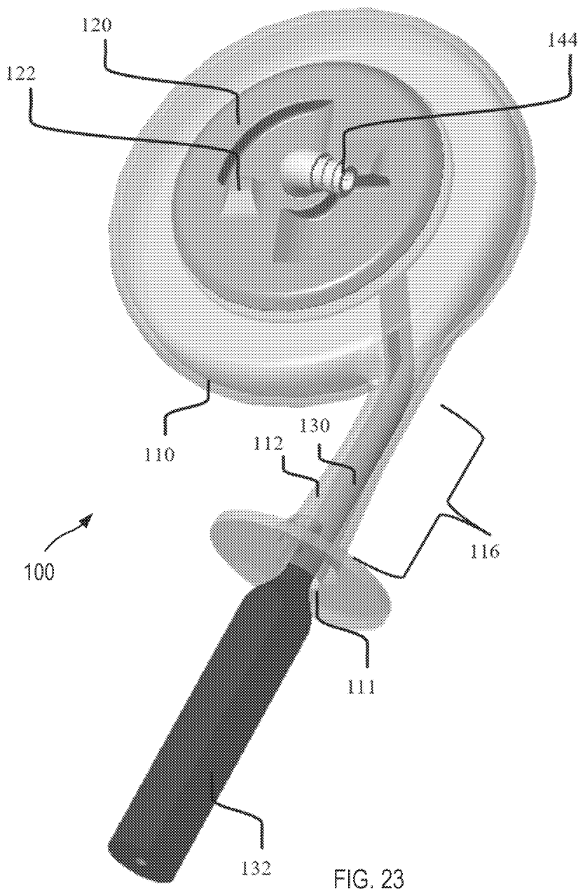

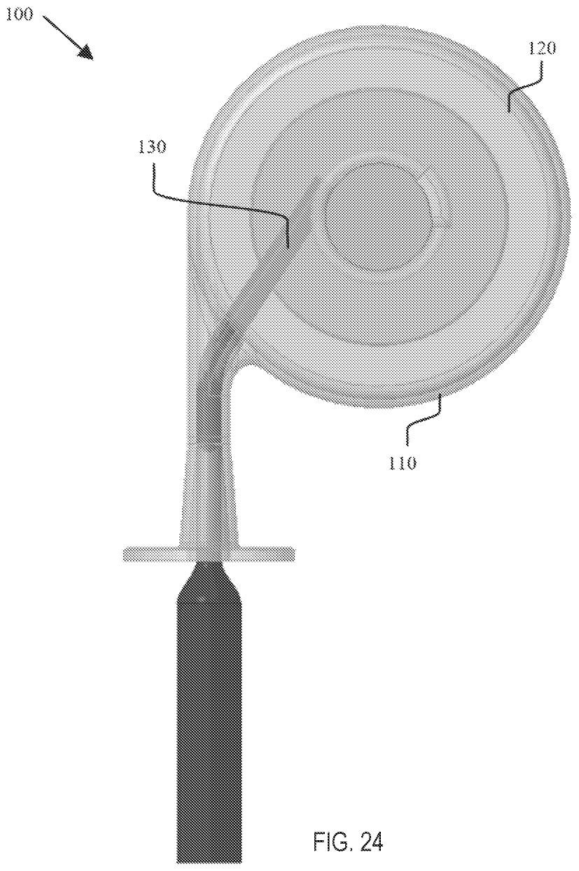

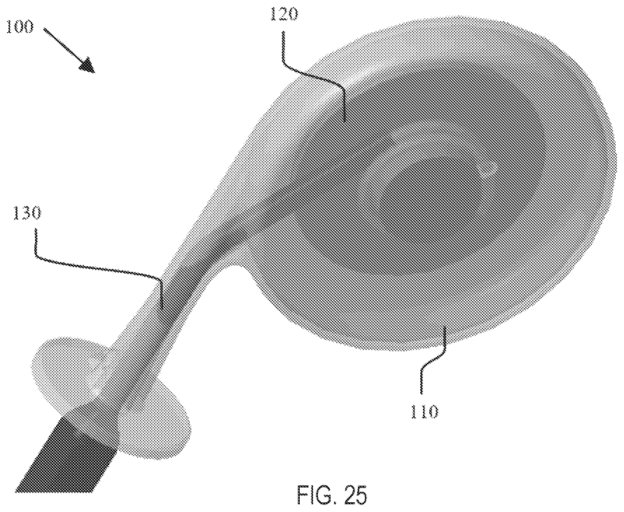

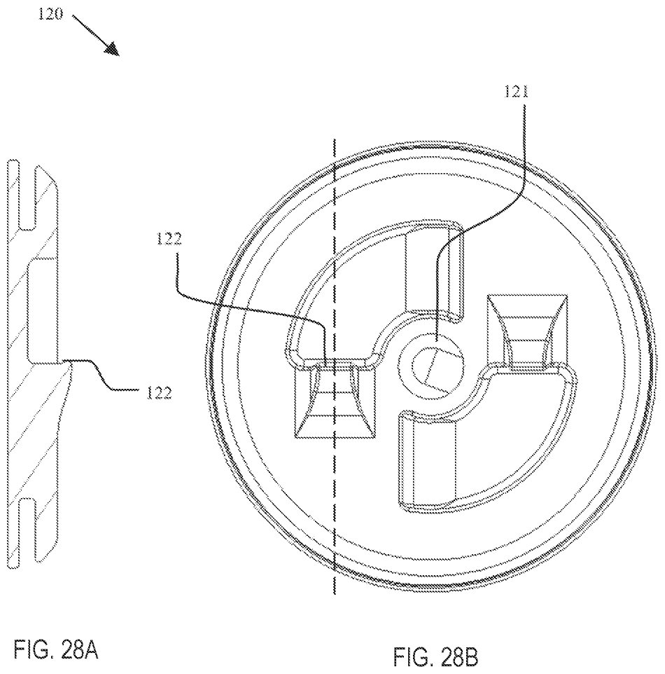

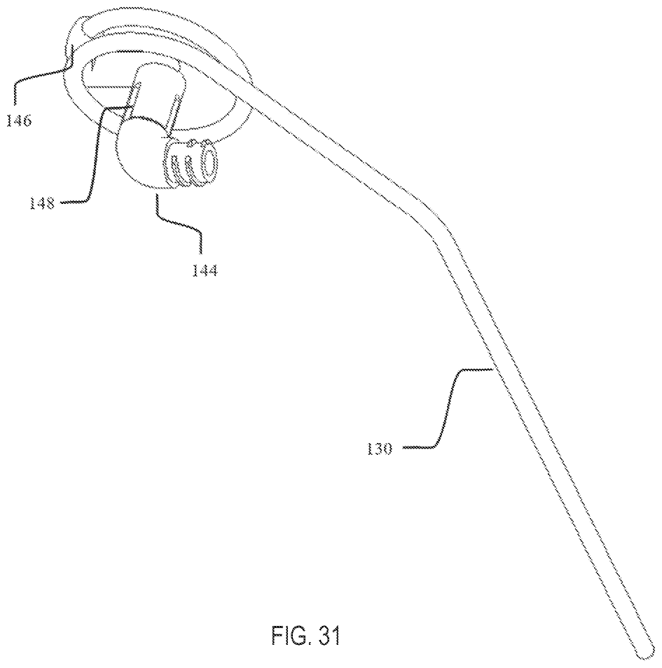

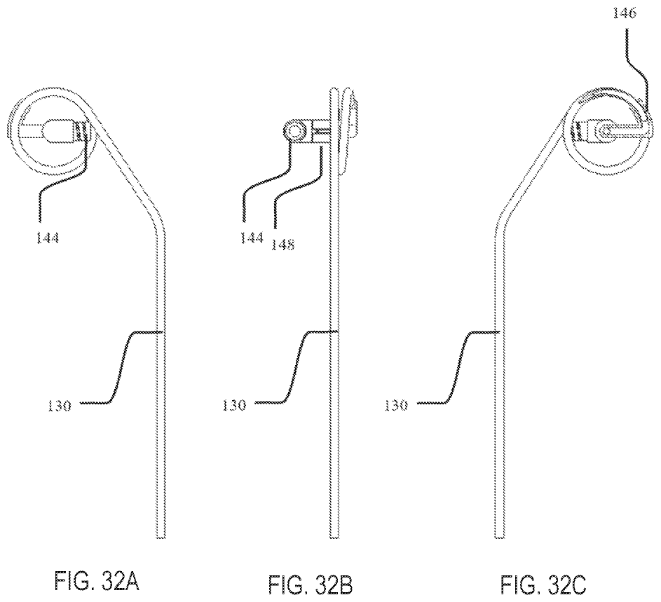

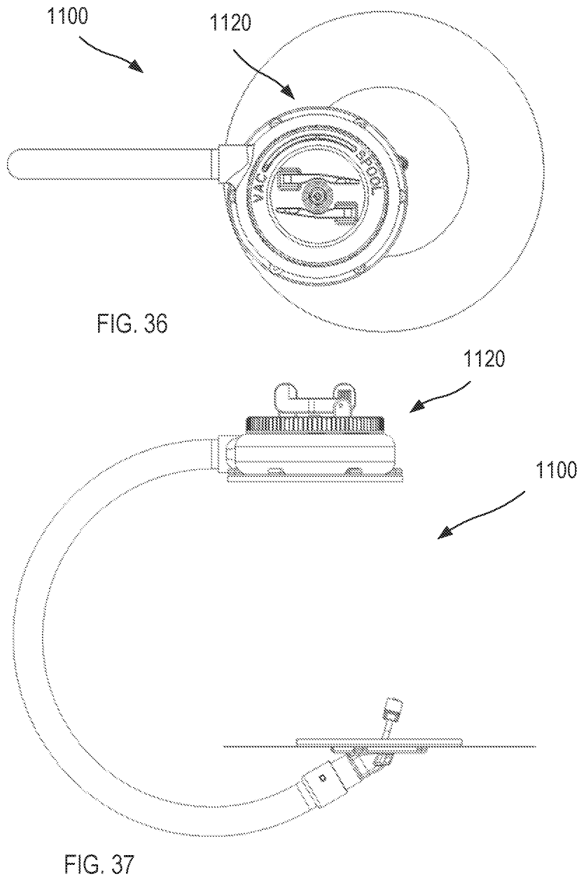

FIGS. 23-35 are a preferred embodiment of a device that may be used for post-operative wound treatment with a rotatable spool and a catheter. In some embodiments, the device is configured to remove material from the closed wound and/or deliver material to the closed wound. In some embodiments, the material removed from the closed wound comprises exudates. In some embodiments, the material delivered to the closed wound comprises antibiotics, irrigation fluid, or any combination thereof. In some cases, any one or more embodiment features described in FIGS. 36-67 can be used on conjunction with or as a replacement for one or more features of the embodiments depicted in FIGS. 23-35.

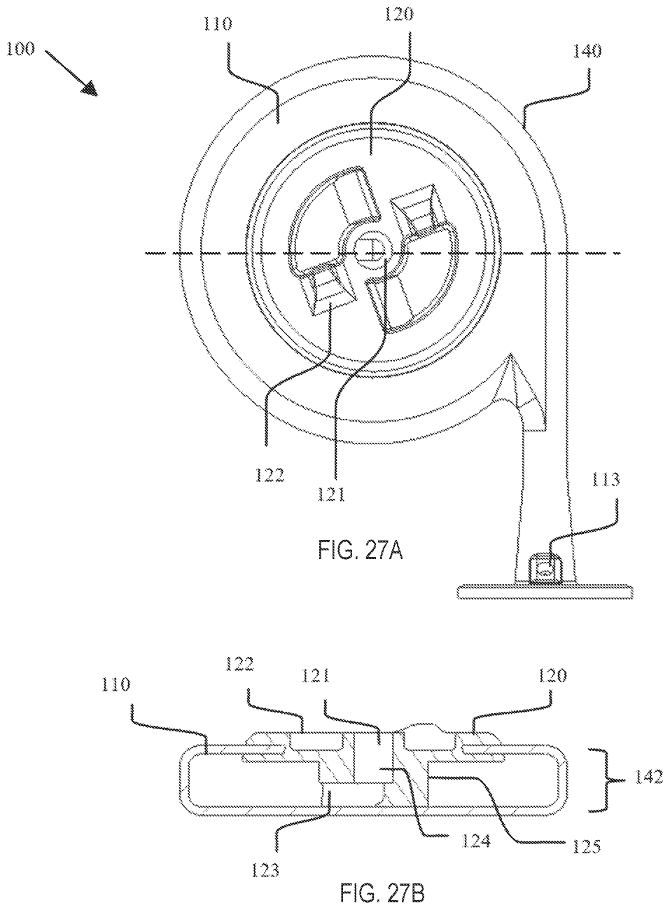

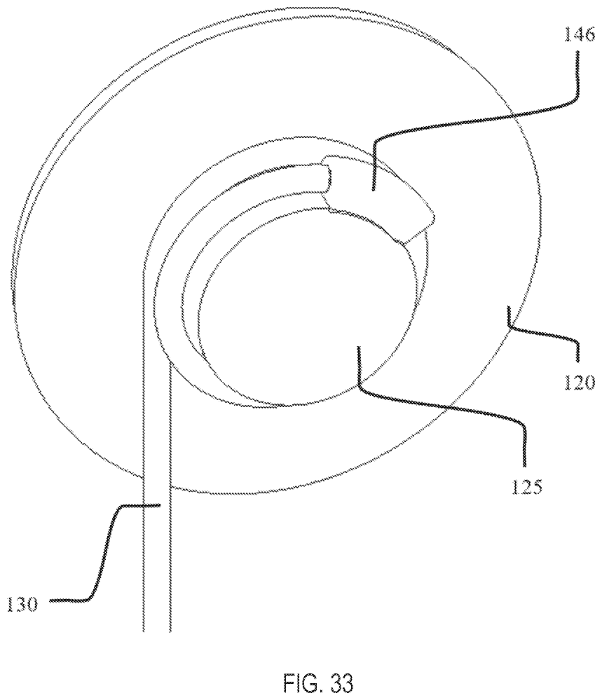

As shown per FIGS. 23-27B, the device 100 comprises a housing 110, a spool 120, and a catheter 130. In some embodiments, the device comprises a cannula or tubing instead of a catheter. In some embodiments, at least a portion of the spool 120 resides within the housing. In some embodiments, the spool 120 is positioned partly within an interior of the housing. Further, as shown, the spool 120 is configured to rotate relative to the housing 110. In some embodiments, the spool 120 is configured to rotate relative to the housing 110 about a single axis. In some embodiments, the spool 120 is configured to rotate relative to the housing 110 about two or more axis. In some embodiments, the spool 120 is configured to rotate relative to the housing 110 such that a water-tight cavity is formed between the spool 120 and the housing 110. In some embodiments, at least a portion of the spool 120 is configured to rotate relative to the housing 110. In some embodiments, the entire spool 120 rotates relative to the housing 110. In some embodiments, the housing 110 comprises a bearing, an O-ring, a seal, or any combination thereof to enable the spool 120 to rotate within the housing. In some embodiments, per FIGS. 27A, 27B, and 33, rotating the spool 120 in a first direction with respect to the housing 110 winds the catheter 130 into the housing 110 and onto or about a column 125. In some embodiments, rotating the spool 120 in a second direction opposite the first direction unwinds the catheter 130 from the column. In some embodiments, rotating the spool 120 in a first direction relative to the housing 110 translates at least a portion of the catheter 130 into the housing 110, and wherein rotating the spool 120 in a second direction opposite the first direction translates at least a portion of the catheter 130 out of the housing 110.

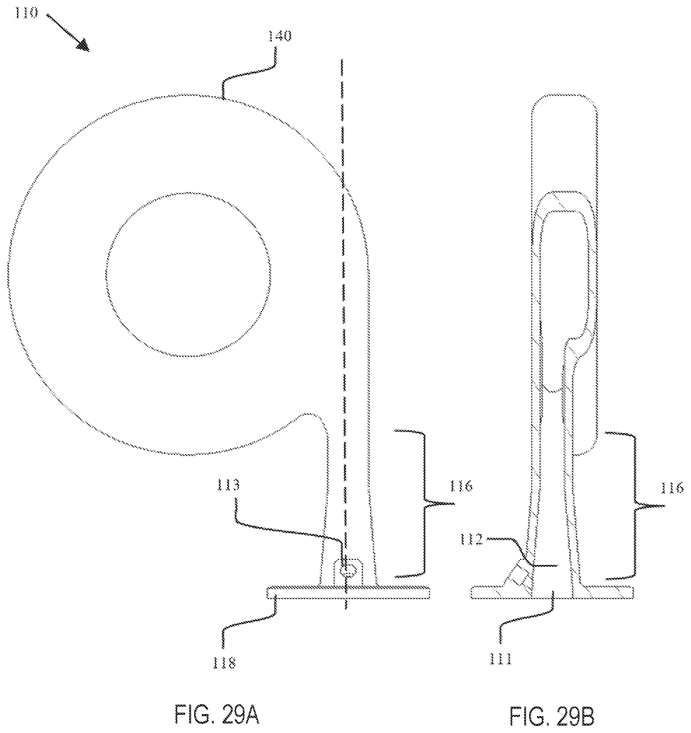

As shown in FIGS. 29A and/or 29B, the housing 110 comprises an aperture 111 on its outer surface and a channel 112 extending from the aperture 111 towards an interior portion of a first portion 140 of the housing. In some embodiments, at least a portion of the channel 112 is tapered. In some embodiments, at least a portion of the channel 112 has a circular cross section, oval cross section, conical cross section, a spiral cross section, a rectangular cross section, or any combination thereof. As shown, in some embodiments, the taper of the channel 112 tapers inwards from the aperture 111. In some embodiments, the taper of the channel 112 is a linear taper. In some embodiments, the taper of the channel 112 is a logarithmic taper. In some embodiments, the channel 112 has a minimum diameter of greater than an outer diameter of the catheter 130. In some embodiments, a first portion 140 of the housing 110 has an outer width or diameter of about 60 mm to about 120 mm, including increments therein. In some embodiments, the housing 110 has an outer height 142 (FIGS. 27A and/or 27B) of about 10 mm to about 25 mm, including increments therein. In some embodiments, the channel 112 has an outer width or diameter of about 5 mm to about 20 mm. In some embodiments, the channel 112 is configured as an elongate shaft, as disclosed herein.

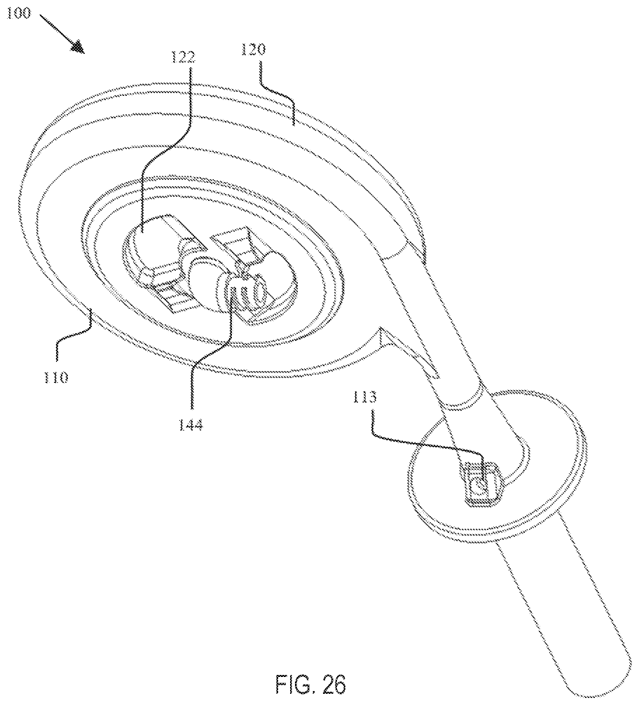

In some embodiments, per FIGS. 26, 27A, 27B, 29A, and/or 29B, the housing 110 further comprises a pressure outlet 113 extending from the outer surface of the housing 110 to an interior of the housing 110. In some embodiments, the pressure outlet 113 is disposed proximal to the channel 112. In some embodiments, the pressure outlet 113 is disposed on an outer surface 116 of the housing along the channel 112. In some embodiments, the pressure outlet 113 is disposed on an outer surface 116 of the housing along the channel 112 proximal to the aperture 111. In some embodiments, the pressure outlet 113 is configured to receive a connector. In some embodiments, the pressure outlet 113 is configured to receive a luer connector. In some embodiments, the pressure outlet 113 enables NPWT via connection with a NPWT system. In some embodiments, the pressure outlet 113 is configured to remove material, such as exudates, from the closed wound.

As shown in FIGS. 29A and/or 29B, in some embodiments, the outer housing proximal to the aperture 111 further comprises a flange 118. In some embodiments, flange 118 is disposed about the aperture 111. In some embodiments, an adhesive pad, plastic film, or any suitable cover is disposed about and/or over the flange 118 to form an airtight seal between the flange and a person's skin surrounding the closed wound. In some embodiments, flange 118 has a maximum outer width or diameter of about 20 mm to about 80 mm, including increments therein.

Per any one or more of FIGS. 26-28B and 31-33, the spool 120 comprises a column 125, a source opening 121, a catheter opening 123, and a conduit 124 between the source opening 121 and the catheter opening 123. In some embodiments, the source opening 121 is on an outer surface of the spool 120. As shown, the source opening 121 is disposed on, and concentric within, the surface of the spool 120. In some embodiments, the source opening 121 is disposed on an outer surface of the spool 120, spaced apart from the center and/or rotational axis of said spool. In some embodiments, the catheter opening 123 is disposed on an inner surface of the spool 120. In some embodiments, the catheter opening 123 is disposed on an inner surface of a spool flange. In some embodiments, the catheter opening 123 is disposed on an inner surface of the column 125. In some embodiments, the conduit 124 is within the column 125. In some embodiments, the column 125 and the conduit 124 are concentric. In some embodiments, the source opening 121, the catheter opening 123, or both comprises a luer connector. In some embodiments, the source opening 121 and/or catheter opening 123 comprise of any type of connection or connector disclosed herein. In some embodiments, at least one of the source opening 121 and the catheter opening 123 comprise a male luer connector, a female luer connector, or both. In some embodiments, at least one of the source opening 121 and the catheter opening 123 comprise two or more luer connectors. In some embodiments, at least one of the source opening 121 and the catheter opening 123 comprise a bent connector. In some embodiments, at least one of the source opening 121 and the catheter opening 123 comprise a 90-degree bent connector. In some embodiments, at least one of the source opening 121 and the catheter opening 123 comprise a straight connector. In some embodiments, the spool 120 has an outer height of about 10 mm to about 20 mm. In some embodiments, the spool 120 has an outer width or diameter of about 40 mm to about 80 mm. In some embodiments, the spool 120 comprises a unitary construction, including the column 125. In some embodiments, the spool 120 comprises the column 125 removably coupled to a spool flange.

In some embodiments, the conduit 124 is disposed between the source opening 121 and the catheter opening 123. In some embodiments, the column 125 extends from the bottom surface of the spool 120. In some embodiments, the source opening 121, the catheter opening 123, the column 125, or any combination thereof is generally concentric to an axis of symmetry of the spool 120. In some embodiments, the source opening 121, the catheter opening 123, the column 125, or any combination thereof is generally perpendicular to an axis of symmetry of the spool 120. In some embodiments, the source opening 121, the catheter opening 123, the column 125, or any combination thereof are generally concentric to each other. In some embodiments, the source opening 121, the catheter opening 123, the column 125, or any combination thereof are generally perpendicular to each other. In some embodiments, the column 125 has a maximum outer width or diameter of about 15 mm to about 40 mm, including increments therein.

In some embodiments, per FIGS. 26-28B, the outer surface of the spool 120 comprises a torque grip 122. In some embodiments, the torque grip 122 comprises a depression, a protuberance, or both. In some embodiments, the torque grip 122 enables a user to rotate the spool 120 relative to the housing 110 in one or more rotational directions. In some embodiments, the top surface of the spool 120 comprises a handle and/or protrusion to rotate the spool. In some embodiments, the top surface of the spool 120 is coupled with an electric motor for automated rotation of the spool 120. In some embodiments, at least a portion of the spool contacts an inner surface of the housing when the spool rotates within the housing 110. In some embodiments, at least a portion of the spool contacts an inner surface of the housing when the spool rotates within the housing 110 to prevent the housing 110 from collapsing under negative pressure. In some embodiments, a flange in the spool 120, the housing 110, or both enables the spool 120 to rotate relative to the housing 110. In some embodiments, a groove in the spool 120, the housing 110, or both enables the spool 120 to rotate relative to the housing 110. In some embodiments, the spool 120 further comprises a biasing element that biases a spring in a first direction with respect to the housing 110. In some embodiments, the biasing element comprises as spring, a piston, a weight, or any combination thereof.

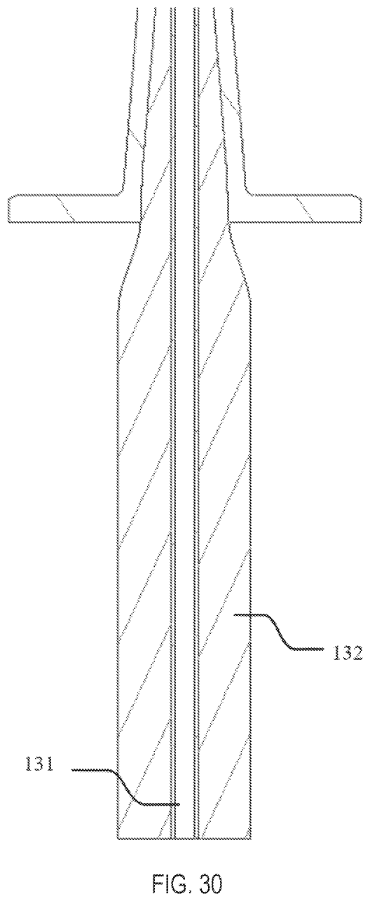

Per FIGS. 30-33, the catheter 130 has a distal portion, a proximal portion, and defines a passageway 131 therebetween. In some embodiments, per FIG. 33, a terminus of the proximal portion of the catheter 130 is configured to couple to the catheter opening 123 of the spool 120. In some embodiments, the terminus of the proximal portion of the catheter 130 is configured to couple to the catheter opening 123 of the spool 120 via a catheter connector 146. In some embodiments, the catheter connector 146 is at least partially positioned within the catheter opening 123. In some embodiments, the catheter connector extends from the catheter opening 123. In some embodiments, a source connector 144 is at least partially positioned within the source opening 121. In some embodiments, the source connector extends from the source opening 121. In some embodiments, at least one of the source connector 144 and the catheter connector 146 is a luer connection. In some embodiments, a connector passageway 148 is disposed between the source connector 144 and catheter connector 146. In some embodiments the source connector 144, the catheter connector 146, and the passageway connector 148 are at least partially positioned within the spool 121. In some embodiments the source connector 144, the catheter connector 146, and the passageway connector 148 are at least partially positioned within the column 125. In some embodiments, the catheter 130 is configured to wrap around the column 125 of the spool 120. In some embodiments, the catheter 130 wraps 1, 2, 3, 4, 5, 6, 7, 8, 9, 10 or more times around the column 125 of the spool 120. In some embodiments, the terminus of the proximal portion of the catheter is configured to removably couple to the catheter opening 123 of the spool 120. In some embodiments, the terminus of the Proximal portion of the catheter is configured to permanently couple to the catheter opening 123 of the spool 120. In some embodiments, the distal portion of the catheter 130 is configured to be inserted through the aperture 111 of the housing 110 and into the closed wound. In some embodiments, the passageway 131 of the catheter 130 is in fluid communication with the closed wound. In some embodiments, the passageway 131 of the catheter 130 and the source opening 121 are in two-way fluidic communication. In some embodiments, the passageway 131 is configured to remove a material from the closed wound via a negative pressure source coupled to the source opening 121. In some embodiments, the passageway 131 is configured to remove exudates from the closed wound. In some embodiments, the passageway 131 is configured to deliver a material to the closed wound via a delivery source coupled to the source opening 121. In some embodiments, the passageway 131 is configured to deliver antibiotics to the closed wound. In some embodiments, the passageway 131 is configured to provide irrigation to the closed wound.

As shown in FIGS. 23 and 30, the device further comprises a wound dressing 132. The wound dressing 132 may be the same as those described in FIGS. 1A, 1B, and/or 1C. In some embodiments, the wound dressing 132 is disposed about the distal portion of the catheter 130. In some embodiments, a proximal of the catheter 130 is inserted within the wound dressing. In some embodiments, the catheter is configured to retract the wound dressing 132 from the closed wound through the aperture 111 and into the tapered portion 112 when the catheter is being wrapped about the column 125. In some embodiments, the wound dressing 132 is configured to be compressed through the tapered portion 112. In some embodiments, the catheter is affixed to the wound dressing 130.

In some embodiments, the catheter 130 comprises one or more perforations between an outer surface of the catheter 130 and an inner surface of the catheter 130. In some embodiments, the one or more perforations are disposed about the distal portion of the catheter 130. In some embodiments, such perforations enable fluids and/or other materials enter the catheter 130. In some embodiments, such perforations enable fluids and/or other materials to exit from the catheter 130 and into the closed wound. In some embodiments, the fluids and/or materials must pass through the wound dressing to enter the catheter from the closed wound, and/or to enter the closed wound from the catheter. In some embodiments, the wound dressing comprises a porous foam. In some embodiments, the device further comprises a mesh element disposed about the wound dressing 132. In some embodiments, the mesh element is configured to capture any portion of the wound dressing that separates into the closed wound. In some embodiments, the mesh element is configured as a removal element as described in FIGS. 1A, 1B, and/or 1C.

Also provided herein is a post-operative surgical site wound treatment device, the device comprising: a housing comprising: an aperture in an outer surface of the housing; a channel extending from the aperture to an interior surface of the housing, wherein at least a portion of the channel is tapered; a spool configured to rotate relative to the housing, wherein at least a portion of the spool resides within the housing, the spool comprising: a column; a source opening on an outer surface of the spool; a catheter opening on an inner surface of the spool; and a conduit between the source opening and the catheter opening; and a catheter having a distal portion and a proximal portion, wherein the proximal portion wraps around the column of the spool, wherein a terminus of the proximal portion couples to the catheter opening of the spool, and wherein the distal portion is inserted through the aperture of the housing and into the closed wound.

In some embodiments, at least one of the housing 110, the spool 120, and the catheter 130, are formed of plastic, metal, carbon fiber, fiberglass, or any combination thereof. In some embodiments, at least one of the housing 110, the spool 120, and the catheter 130, is transparent. In some embodiments, at least one of the housing 110, the spool 120, and the catheter 130, is translucent. In some embodiments, at least one of the housing 110, the spool 120, and the catheter 130, is opaque. In some embodiments, at least one of the housing 110, the spool 120, and the catheter 130, are formed by stamping, blow molding, injection molding, or any combination thereof.

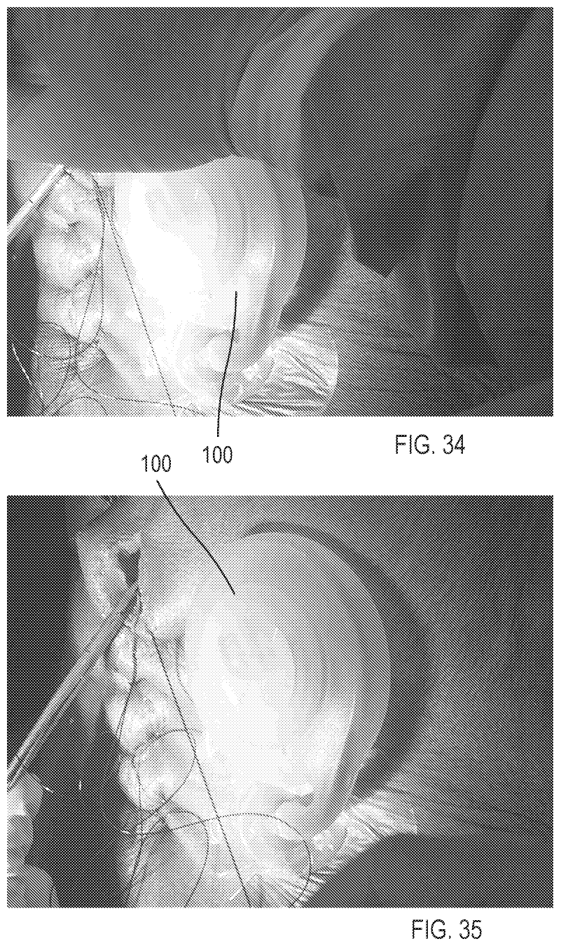

FIGS. 34 and 35 shows images of the post-operative surgical site wound treatment device for transferring a material through a closed wound.

Also provided herein is a method of post-operative surgical site treatment through a closed wound, the method comprising: providing the device described in FIGS. 23-33; inserting the catheter into the closed wound; closing the wound about the catheter, and delivering an antibiotic and/or irrigation fluids, and/or suctioning exudates and/or other material from the closed wound. In some embodiments, the method further comprises rotating the spool in a first direction, thereby unwinding the catheter from the column and extending through the aperture and into the closed wound. In some embodiments, the antibiotic and/or irrigation fluids are delivered to the closed wound through the source opening and via the catheter. In some embodiments, the method comprises coupling an antibiotic source and/or an irrigation fluid source to the source opening. In some embodiments, the source opening comprises a luer connection that is configured to couple with the antibiotic source and/or irrigation fluid. In some embodiments, the exudates are suctioned and removed from the closed wound through the source opening (via the catheter) and/or the pressure outlet. In some embodiments, a NPWT (negative-pressure wound therapy) system is applied to the device. In some embodiments, the method comprises coupling the NPWT (negative-pressure wound therapy) system and/or other suctioning device to the catheter opening and/or to the pressure outlet. In some embodiments, the catheter opening and/or pressure outlet comprises a connector, e.g., nozzle, luer connection, that is configured to couple with the NPWT system and/or other suctioning device. In some embodiments, the method further comprises removing the catheter from the closed wound by rotating the spool. In some embodiments, the method further comprises rotating the spool in a second direction, thereby retracting the catheter into the housing and winding the catheter about the column. In some embodiments, the device comprises a wound dressing disposed about a first portion of the catheter, wherein the wound dressing and the first portion of the catheter are inserted into the wound. In some embodiments, the method further comprises removing the catheter and wound dressing from the closed wound by rotating the spool in a second direction, thereby enabling the catheter to wind about or onto the column. In some embodiments, the method further comprises retracting the wound dressing into the tapered portion by rotating the spool in a second direction.

FIGS. 36 and 37 depict aspects of a post-operative wound treatment device 1100 according to embodiments of the present invention. In these figures, a spool 1120 of the device 1100 is in a vacuum (VAC) mode.

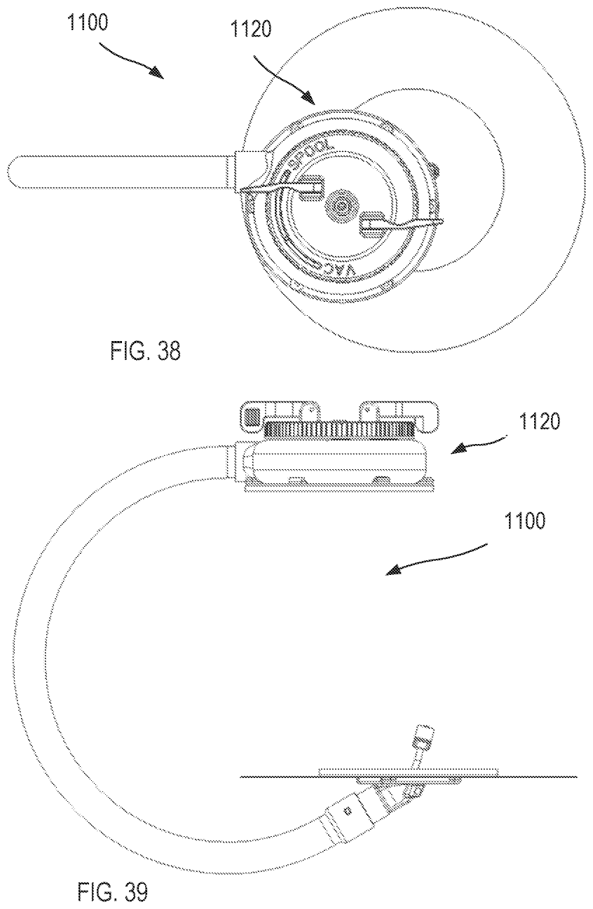

FIGS. 38 and 39 depict aspects of a post-operative wound treatment device 1100 according to embodiments of the present invention. In these figures, a spool 1120 of the device 1100 is in a spooling (spool) mode.

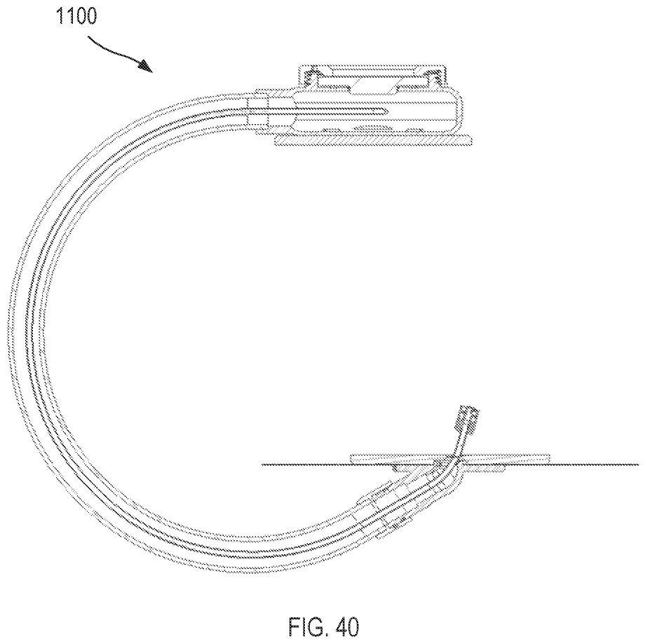

FIG. 40 provides a section view of a post-operative wound treatment device 1100 according to embodiments of the present invention.

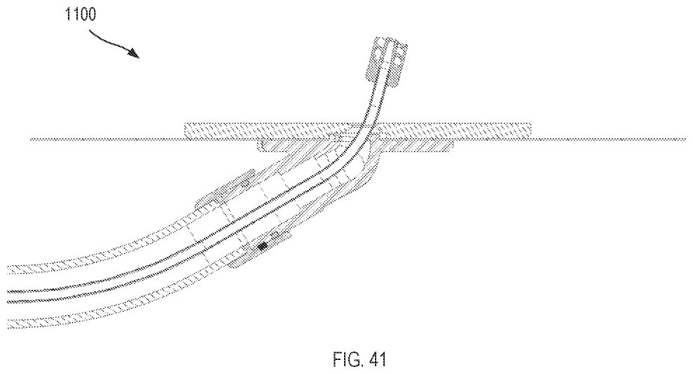

FIG. 41 provides an incision site section view of a portion of a post-operative wound treatment device 1100 according to embodiments of the present invention.

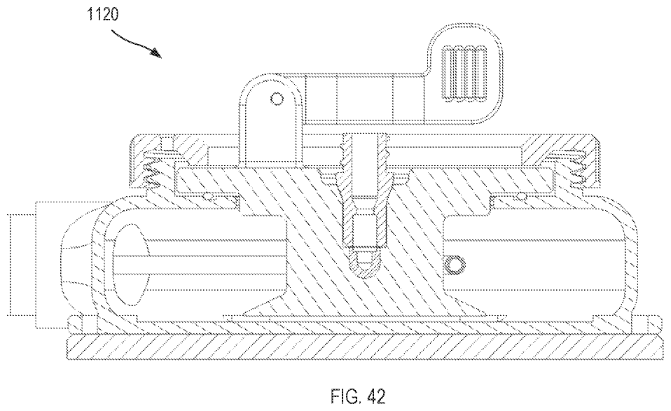

FIG. 42 provides a section view of a spool 1120 of a post-operative wound treatment device according to embodiments of the present invention.

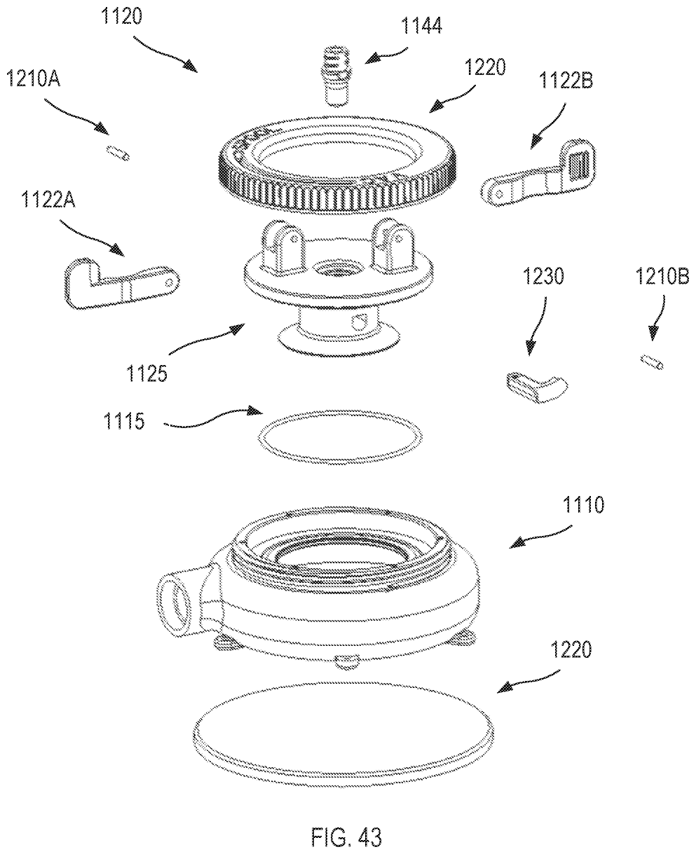

FIG. 43 provides an exploded view of a spool 1120 of a post-operative wound treatment device according to embodiments of the present invention. As shown here, the spool 1120 includes a luer connector or source connector 1144, pins 1210A and 1210B (e.g. M2X10 dowel pins), a spool lid 1220, spool levers 1122A and 1122B, a spool element or spool column 1125, and a tube elbow connector 1230.

The spool 1120 can be configured to rotate relative to a housing 1110. In some embodiments, at least a portion of the spool 1120 is configured to rotate relative to the housing 1110. In some embodiments, the entire spool 1120 rotates relative to the housing 1110. In some embodiments, the housing 1110 includes a rotational mechanism 1115 such as a bearing, an O-ring, a seal, or any combination thereof, to enable the spool 1120 to rotate within the housing. As shown here, the rotational mechanism 1115 can be an O-ring. A post-operative wound treatment device can also include a spool pad 1220.

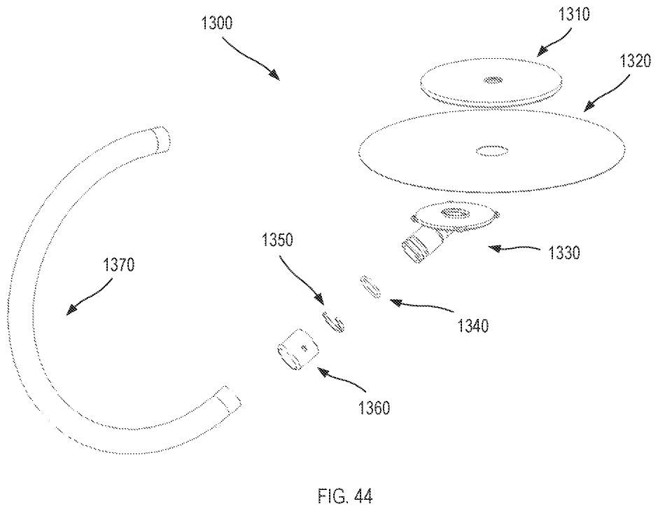

FIG. 44 provides an exploded view of an incision site mechanism 1300 of a post-operative wound treatment device according to embodiments of the present invention. As shown here, the incision site mechanism 1300 includes a dressing pad 1310, a dressing seal 1320, an incision site connector 1330, an O-ring 1340, a C-clip 1350, a vacuum hose fitting 1360, and a vacuum hose 1370.

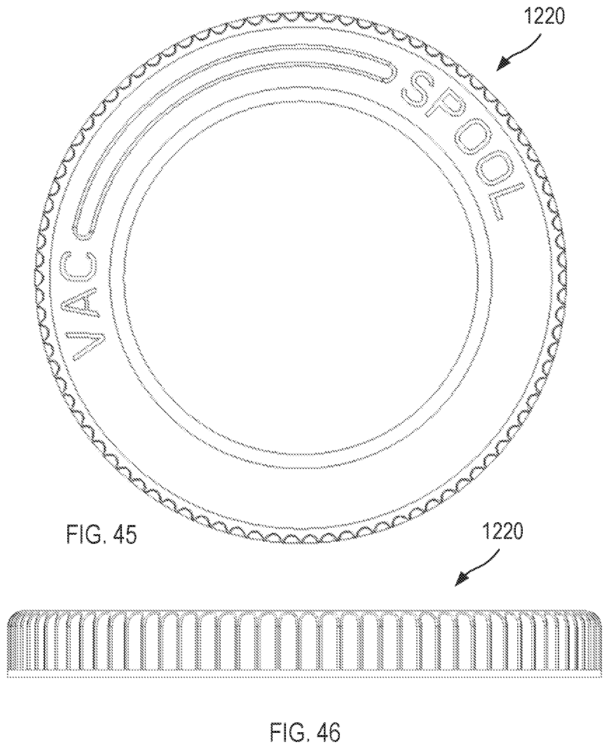

FIGS. 45 and 46 depict aspects of a spool lid 1220 of a post-operative wound treatment device, according to embodiments of the present invention.

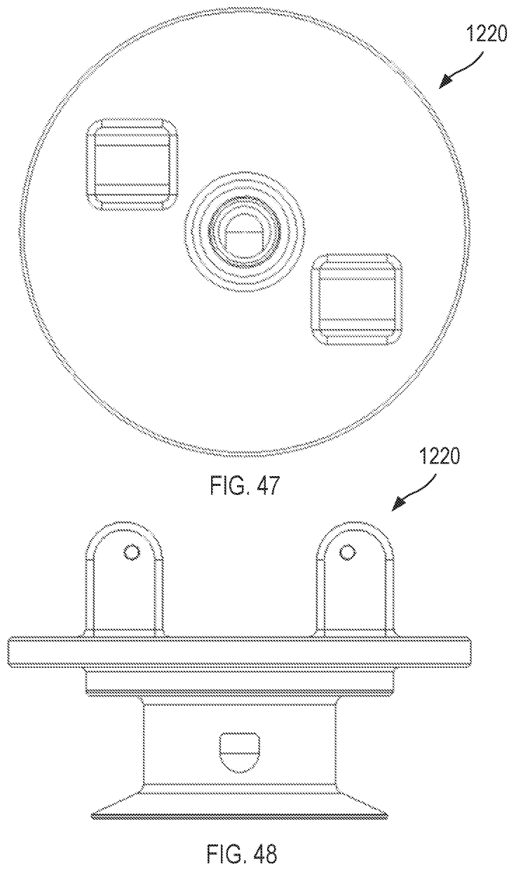

FIGS. 47 and 46 depict aspects of a spool element or spool column 1125 of a post-operative wound treatment device, according to embodiments of the present invention.

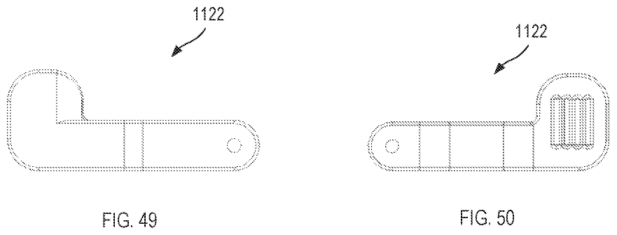

FIGS. 49 and 50 depict aspects of a spool lever 1122 of a post-operative wound treatment device, according to embodiments of the present invention.

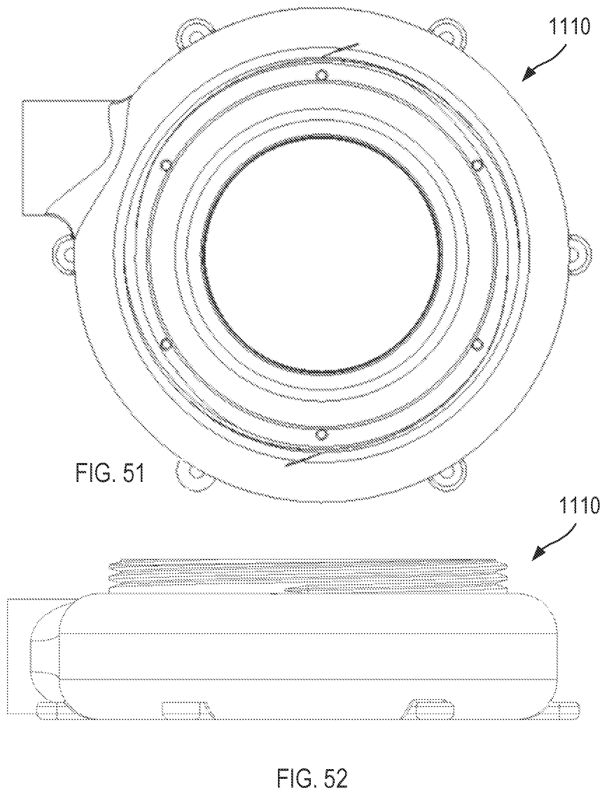

FIGS. 51 and 52 depict aspects of a spool housing 1110 of a post-operative wound treatment device, according to embodiments of the present invention.

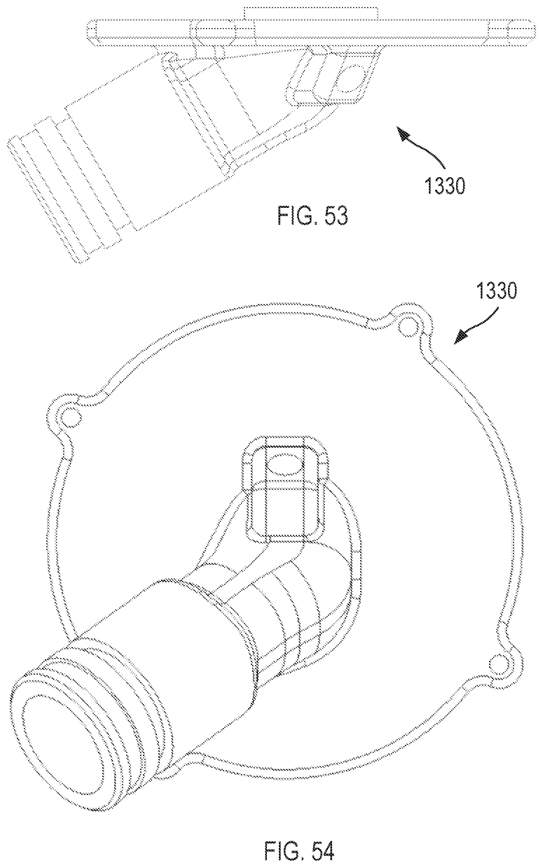

FIGS. 53 and 54 depict aspects of an incision site connector 1330 of a post-operative wound treatment device, according to embodiments of the present invention.

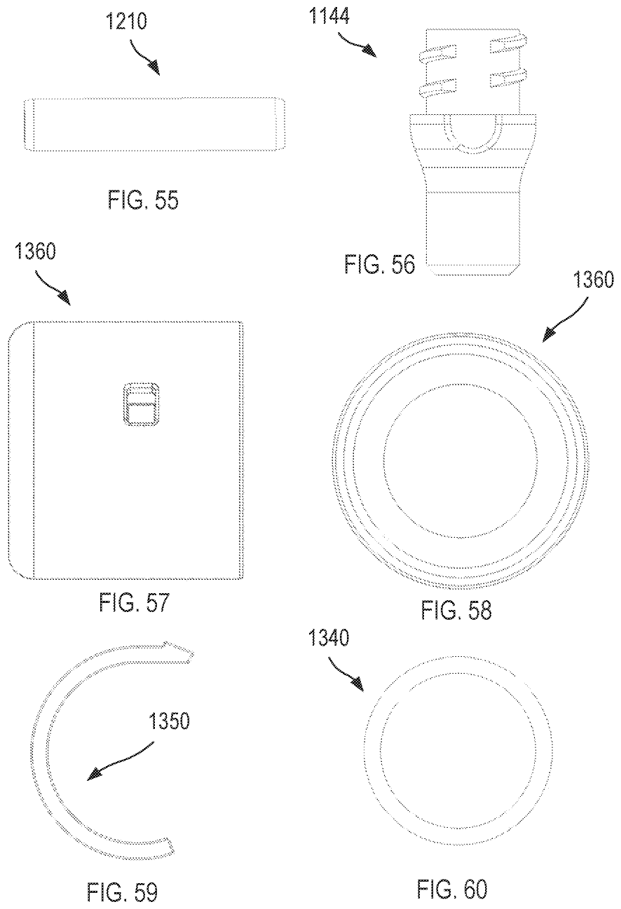

FIG. 55 depicts aspects of a pin 1210 of a post-operative wound treatment device, according to embodiments of the present invention.

FIG. 56 depicts aspects of a luer connector or luer fitting 1144 (female) of a post-operative wound treatment device, according to embodiments of the present invention.

FIGS. 57 and 58 depict aspects of a vacuum hose fitting 1360 of a post-operative wound treatment device, according to embodiments of the present invention.

FIG. 59 depicts aspects of a C-clip 1350 of a post-operative wound treatment device, according to embodiments of the present invention.

FIG. 60 depicts aspects of an O-ring 1340 of a post-operative wound treatment device, according to embodiments of the present invention.

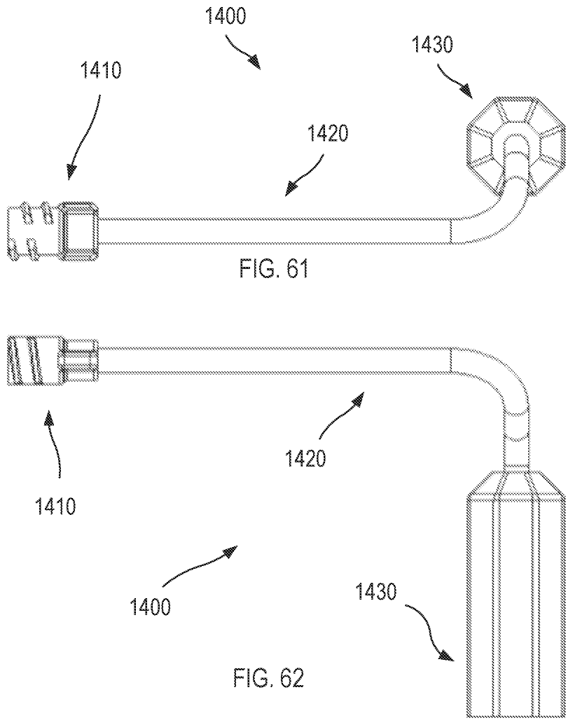

FIGS. 61 and 62 depict aspects of a foam assembly 1400 of a post-operative wound treatment device, according to embodiments of the present invention. As shown in these figures, a foam assembly 1400 can include a luer connector 1410 (e.g. female luer connector), a catheter 1420 (e.g. antibiotics catheter), and a foam element 1430 (e.g. hexagonal foam).

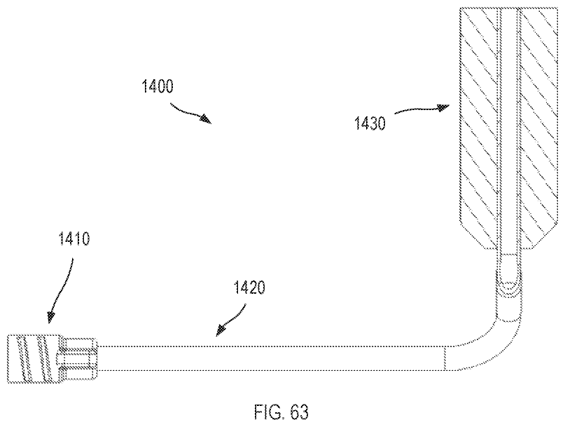

FIG. 63 depicts aspects a cross section view of a foam assembly 1400 of a post-operative wound treatment device, according to embodiments of the present invention. As shown here, a foam assembly 1400 can include a luer connector 1410 (e.g. female luer connector), a catheter 1420 (e.g. antibiotics catheter), and a foam element 1430 (e.g. hexagonal foam).

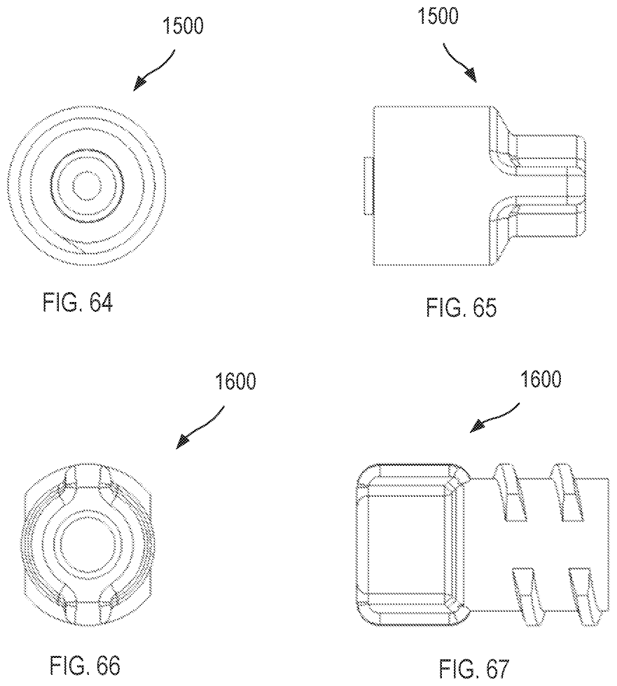

FIGS. 64 and 65 depict aspects of a luer connector 1500 (e.g. male luer connector) of a post-operative wound treatment device, according to embodiments of the present invention.

FIGS. 66 and 67 depict aspects of a luer connector 1600 (e.g. female luer connector) of a post-operative wound treatment device, according to embodiments of the present invention.

Although the preceding description contains significant detail in relation to certain preferred embodiments, it should not be construed as limiting the scope of the invention but rather as providing illustrations of the preferred embodiments.

Embodiments of the present invention encompass kits having one or more components of a post-operative surgical site wound treatment device as disclosed herein. In some embodiments, the kit includes one or more post-operative surgical site wound treatment device components, along with instructions for using the device(s) for example according to any of the methods disclosed herein.

All features of the described systems and devices are applicable to the described methods mutatis mutandis, and vice versa.

In addition, each reference provided herein in incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-39. (canceled)

40. A post-operative surgical site wound treatment device, the device comprising:
a housing having an aperture in an outer surface of the housing, and a channel extending from the aperture to an interior surface of the housing;
a spool configured to rotate relative to the housing, wherein at least a portion of the spool resides within the housing; and
a wound dressing coupled with the spool, wherein rotation of the spool retracts the wound dressing from a closed wound.

41. The device of claim 40, wherein the housing further comprises a pressure outlet extending from the outer surface of the housing to an interior of the housing.

42. The device of claim 41, wherein the pressure outlet is disposed proximal to the channel.

43. The device of claim 41, wherein the pressure outlet is disposed on the channel.

44. The device of claim 40, wherein the outer surface of the housing further comprises a flange.

45. The device of claim 44, wherein the flange is disposed about the aperture.

46. The device of claim 40, wherein an outer surface of the spool comprises a torque grip.

47. The device of claim 46, wherein the torque grip comprises a depression, a protuberance, or both.

48. The device of claim 40, wherein the channel is within a column of the spool.

49. The device of claim 40, further comprising a catheter.

50. The device of claim 49, wherein the catheter comprises one or more perforations between an outer surface of the catheter and an inner surface of the catheter.

51. The device of claim 50, wherein the one or more perforations are disposed about the distal portion of the catheter.

52. The device of claim 40, wherein a flange in the spool, the housing, or both enables the spool to rotate relative to the housing.

53. The device of claim 40, wherein a groove in the spool, the housing, or both enables the spool to rotate relative to the housing.

54. The device of claim 40, wherein rotating the spool in a first direction relative to the housing translates the wound dressing toward the housing and rotating the spool in a second direction opposite the first direction translates the wound dressing away from the housing.

55. The device of claim 40, wherein at least a portion of the spool contacts an inner surface of the housing when the spool rotates within the housing.

56. The device of claim 40, wherein the wound dressing disposed about a distal portion of a catheter.

57. The device of claim 40, further comprising a mesh disposed about the wound dressing.

58. The device of claim 40, wherein at least a portion of the channel is tapered.

59. The device of claim 40, wherein the wound dressing is coupled with a tether.

60. The device of claim 40, wherein the spool further comprises a source opening on an outer surface of the spool, a catheter opening on the inner surface of the spool, and a conduit between the source opening and the catheter opening, and wherein a terminus of the proximal portion of a catheter couples to the catheter opening of the spool.

61. The device of claim 60, wherein the catheter and the source opening are in two-way fluidic communication.
